(19) United States
(12) Patent Application Publication
(10) Pub. No.: US 2026/0038659 A1
(43) Pub. Date: Feb. 5, 2026

(54) METHODS AND APPARATUS FOR GENERATING AN INDICATOR OF SMOKING HISTORY

(71) Applicant: TidalSense Limited, Cambridge (GB)

(72) Inventors: Ameera Patel, Cambridge (GB); Henry Alexander Broomfield, Cambridge (GB); Rui Hen Lim, Cambridge (GB)

(21) Appl. No.: 19/306,385

(22) Filed: Aug. 21, 2025

Related U.S. Application Data
(63) Continuation of application No. PCT/GB2024/050480, filed on Feb. 21, 2024.

(30) Foreign Application Priority Data
Feb. 21, 2023 (GB) 2302464.9

(51) Int. Cl. G16H 15/00; G16H 50/20

(52) U.S. Cl. CPC G16H 15/00; G16H 50/20

(57) ABSTRACT

A method for generating an indicator of smoking history from one or more capnograms produced from a user, the method comprising: obtaining one or more breath waveforms from the one or more capnograms produced from the user, extracting one or more features from the one or more breath waveforms; generating the indicator of smoking history based on the one or more features.

### CROSS-REFERENCE TO RELATED APPLICATIONS

This application is a continuation-in-part of International Application No. PCT/GB2024/050480, filed Feb. 21, 2024, which in turn claims the priority benefit of United Kingdom Application No. 2302464.9, filed Feb. 21, 2023. Each of these applications is incorporated herein by reference in its entirety for all purposes.

### FIELD OF THE INVENTION

The present disclosure provides a method for generating an indicator of smoking history, a method for training a machine learning model to learn an indicator of smoking history, and an apparatus and computer readable medium for the same.

### BACKGROUND

The global tobacco epidemic is considered as one of the greatest threats to public health, accounting for more than 8 million deaths every year, of which 1.2 million are from exposure to second-hand smoke. The economic costs of tobacco use are substantial and smoking costs the global economy an estimated US$1.4 trillion annually in healthcare expenditure and lost productivity. The health and economic burden of smoking disproportionately affects countries with a low socio-demographic index, with over 80% of the 1.3 billion tobacco users living in low and middle-income countries.

Tobacco smoking is widely recognised as the leading contributor to respiratory diseases; it is a primary etiological factor for the development of chronic obstructive pulmonary disease (COPD) and lung cancer, and it also adversely impacts other respiratory diseases such as asthma, tuberculosis and pneumonia. These diseases collectively account for a large proportion of tobacco-attributable respiratory mortality and morbidity around the world. Numerous cross-sectional and longitudinal studies have demonstrated that tobacco smoking has an adverse effect on lung function and respiratory health, although the severity and extent of these changes vary across individuals.

Capnography is a widely used method for the measurement of the partial pressure of exhaled carbon dioxide, especially in critical care and anaesthetics. Continuous monitoring of carbon dioxide levels provides a valuable insight into a user's ventilation and airway potency, and capnography has become an increasingly popular alternative technique to assess pulmonary health. Cambridge Respiratory Innovations' (CRI) N-Tidal device has made it possible to measure CO2 concentration reliably and non-invasively at the mouth, closer to the airways, and with a greater sampling frequency than previously possible, making the technique an appealing alternative to current respiratory diagnostic tools such as spirometry.

Up until now, it has proven to be difficult to determine the effects of lifetime smoking exposure. It would be advantageous to provide a technique of determining the severity of the damage caused by smoking, such as the severity of related diseases, which could then be used to inform treatment choices and to track changes in cardiorespiratory conditions due to smoking cessation.

An objective of the present disclosure is therefore to use capnography data to determine the smoking history of a user, including tobacco smoking, cannabis use, and vaping.

### SUMMARY OF INVENTION

According to a first aspect, the present disclosure provides a method for generating an indicator of smoking history from one or more capnograms produced from a user, the method comprising: obtaining one or more breath waveforms from the one or more capnograms produced from the user; extracting one or more features from the one or more breath waveforms; generating the indicator of smoking history based on the one or more features.

This method allows a user's smoking history to be estimated by generating an indicator of this smoking history. While there is often a link between smoking history and cardiorespiratory disease, it is advantageous to determine a user's smoking history in a way which is decoupled from indicators of cardiorespiratory disease. For example, many regular smokers do not exhibit any signs of cardiorespiratory disease. Notably, this method does not require smoking history to be determined based on user reporting, which is notoriously inaccurate.

The generated indicator could be used in a variety of ways, including to inform decisions for managing a user's cardiorespiratory condition, such as by prescribing treatment options, or to track the efficacy of smoking cessation and/or of treatment(s) (including tracking the reversal of any damage to a user's cardiorespiratory system). Other uses of the generated indicator include estimating the severity of damage caused by smoking and tracking the adherence of a user to a prescribed treatment.

While the breath waveforms could be obtained from volumetric capnogram data, the one or more capnograms preferably comprise time series capnogram data.

One suitable indicator which is used in preferable embodiments of the present invention is ‘lung age’, which compares the condition of a user's cardiorespiratory system with other cohorts of users. Any cohort could be chosen as a reference population. For example, the user could be compared with other people sharing one or more characteristics with the user, such as age, sex, disease, or any other demographic information. Another would be to compare the condition of a user's cardiorespiratory system with the cohort of non-smokers in the population. In this case, the lung age of a smoker would be the equivalent age of a non-smoker with an equal pulmonary function. For example, a 50 year old smoker who has the same pulmonary function as a 75 year old non-smoker would be said to have a lung age of 75 years. This indicator has been found to reliably indicate the effects of smoke exposure and is therefore particularly preferred as an indicator of smoking history.

By tracking the changes in a user's lung age, it is possible to determine the adherence of a user to a prescribed treatment as well as to track the efficacy of the prescribed treatment. This can also be used to track the rate of progression of a disease.

In preferred embodiments, a series of capnograms may be produced from a user over a predetermined period of time and the lung age of a user may be calculated for each capnogram produced from the user. Statistically significant changes or drifts in the user's lung age may then be used to inform clinical decisions. For example, observation of a decrease in lung age after a treatment course can inform a clinician that continued use may be advantageous. Preferably, only changes exceeding a predetermined threshold are considered. In addition or as an alternative to this approach, observation of no change in lung age after a treatment course could signal that a different intervention is required.

Another suitable indicator is pack years, a metric which quantifies a user's smoking history in terms of the total number of cigarettes smoked during their lifetime, where 1 pack year is defined as having smoked a pack of 20 cigarettes every day for a year, i.e. 7,305 cigarettes.

Other suitable indicators include the total number of years a user has smoked and the number of years since a user has stopped smoking. For example, it has been found that in some contexts the number of years of continuous smoke exposure can be a more reliable indicator of cardiorespiratory condition than pack years.

The generated indicator could be provided at different levels of precision. For example, in the case of pack years, the indicated pack years could be to the nearest year or could be in bands, such as 0 to 10 pack years, 10 to 20 pack years, 20 to 30 pack years, 30 to 40 pack years, and 40+ pack years. Likewise, lung age could be given to the nearest 5 years.

These indicators could be used to indicate the smoking history of a user which includes one or more of tobacco use, cannabis use, and vaping.

In some embodiments, the method will also generate one or more further indicators of smoking history. For example, the method could generate both the pack years for a user as well as that user's lung age.

The terms capnogram and breath record are used interchangeably herein to refer to a continuous capnometry measurement in a single session. For example, the user or operator stopping recording of a user's breathing with a capnometry monitor is the end of a capnogram, while starting to record it again is the beginning of another capnogram. However, capnogram and breath record may also refer to a given time period of continuous CO₂ recording (e.g. in a ventilator circuit).

The skilled person will appreciate that the inspiratory baseline and expiratory baseline may be considered the same baseline of a capnogram/breath record, i.e. a respiratory baseline or a capnogram baseline. The inspiratory baseline and the expiratory baseline are named differently in order to distinguish between (once an individual breath waveform has been isolated) the baseline adjacent the expiratory periods of the breath waveform and the baseline adjacent the inspiratory periods of the breath waveform, as well as to more clearly describe the transition points of a waveform.

Generating the indicator of smoking history preferably comprises using the one or more extracted features as inputs to a trained machine learning model, wherein the trained machine learning model is configured to output the indicator of smoking history.

The trained machine learning model is preferably trained using the same or corresponding extracted features that have been extracted from other capnograms labelled with a corresponding indicator of smoking history. For example, the trained machine learning model will preferably have been trained using a method according to any implementation of the second aspect below. As used herein, the term machine learning is also used to refer to statistical inference. For example, a statistical inference model may be referred to as a machine learning model, and a step of training a machine learning model may refer to training a statistical inference model (or a machine learning model).

Generating the indicator of smoking history preferably further comprises using one or more pieces of demographic information as inputs to the trained machine learning model, wherein the one or more pieces of demographic information may comprise one or more of: age; sex; and ethnicity. As noted above, some indicators of smoking history may be based on comparisons with cohorts of the general population and this demographic information could therefore be used to generate an indicator of smoking history. The features extracted exhibited by the breath waveforms which indicate smoking history could also vary between cohorts. For example, features of the breath waveforms which would indicate a long history of smoking in users under 35 might be considered normal in users over 65.

Generating the indicator of smoking history may also comprise using the time of day that each of the one or more capnograms were produced from the user as an input to the trained machine learning model. It has been found that the change in hormonal levels, such as cortisol levels, throughout the day can impact on breath waveforms produced by a user. Using the time of day that each of the one or more capnograms were produced from the user as an input to the trained machine learning model allows for this to be taken into account.

Another characteristic of a user which may affect which features of a breath waveform can be used to determine smoking history includes if that user has a disease, especially if the user has a cardiorespiratory disease. Therefore, generating the indicator of smoking history preferably further comprises: obtaining a disease label associated with the one or more breath waveforms. Non-limiting examples of disease labels include an indicator that a user has one or more of COPD, asthma, or lung cancer.

This disease label could be used as an input to the trained machine learning model, but in some embodiments different machine learning models are trained to generate an indicator or indicators of smoking history based on the disease label. In such cases, generating the indicator of smoking history further comprises: obtaining a disease label associated with the one or more breath waveforms; and selecting the trained machine learning model from two or more trained machine learning models based on the obtained disease label.

Similarly, demographic information such as sex and ethnicity could be used as inputs to the trained machine learning models or different machine learning models could be trained for different combinations of demographic labels and disease labels. For example, there could be different models for male with COPD, male and healthy, female with COPD, and female and healthy.

Optionally, the method further comprises: determining the variability of the one or more extracted features; and using the variability of the extracted features as an input to the trained machine learning model. The variability of the extracted features may also be used to train the machine learning model to create the classifying function (in some embodiments, the classifying function is a predictive function) according to the second aspect. Other parameters could be determined from the one or more extracted features, such as a metric related to the features over time such as mean, median, standard deviation, a distribution or other suitable similarity score such as cosine similarity or distance. Variability of extracted features may be determined using two or more breath waveforms recorded from the same user. More preferably, the plurality of breath waveforms recorded from the same user comprises at least three breath waveforms recorded from the same user. It has been found that determining variability from at least three breath waveforms provides improved accuracy.

The time period used to determine the variability of extracted features may vary depending on the features being examined and/or the indicator the machine learning model is generating (or being trained to generate). The time period over which the breath waveforms were recorded may be, for example, multiple hours, multiple days, multiple weeks or multiple months. For example, the time period may comprise two or more days, preferably the time period comprises five or more days, and more preferably the time period comprises ten or more days. Nevertheless, high accuracy can still be achieved by determining variation without considering the time period.

Preferably, the plurality of breath waveforms are recorded at substantially regular intervals over the time period. For example, when the time period is five days, the plurality of breath waveforms includes a first breath waveform obtained from a first capnogram recorded on the first of the five days, a second breath waveform obtained from a second capnogram recorded on the second of the five days, a third breath waveform obtained from a third capnogram recorded on the third of the five days, and so on. More preferably, capnograms are recorded two or more times a day in order to assess variability of breath waveforms and extracted features across the day.

In some implementations of the first aspect, the trained machine learning model is further configured to output an indication of the importance of an extracted feature that contributed to the generated indicator of smoking history. The method may include the step of outputting this indication. In this way, the indication may be used to evaluate the trained machine learning model, and to interpret the indication by providing additional context.

According to a second aspect, there is provided a method for training a machine learning model to learn an indicator of smoking history, the method comprising: obtaining a plurality of breath waveforms from a plurality of capnograms; extracting one or more features from the plurality of breath waveforms; obtaining a label for each breath waveform indicating a corresponding smoking history; and using the extracted features of the plurality of breath waveforms and the corresponding labels, training the machine learning model to learn the indicator of smoking history.

The plurality of capnograms used to train the machine learning model may be individual capnograms from a plurality of different users and/or multiple capnograms produced by a user at different times. A plurality of breath waveforms may also be obtained from a single capnogram of the plurality of capnograms.

The step of extracting one or more features from the plurality of breath waveforms results in a featurised breath waveform or capnogram. The steps of obtaining a plurality of breath waveforms and extracting one or more features therefrom may be repeated to obtain extracted features of a plurality of breath waveforms (i.e. producing a plurality of featurised breath waveforms).

In some embodiments of the second aspect, the machine learning model is trained to learn indicators of an indicator of smoking history to create a predictive function based on the labels obtained from each of the plurality of breath waveforms. That is, the step of using the extracted features of the plurality of breath waveforms and the corresponding labels to train the machine learning model to learn the indicator of smoking history comprises: using the extracted features of the plurality of breath waveforms and the corresponding labels, training the machine learning model to learn the indicator of smoking history to create a predictive function.

The first aspect and the second aspect may comprise any of the following implementations.

In examples, the breath waveform may be a digitally sampled signal representing a quantized amplitude signal. The signal may be encoded as an array of floating point samples, each representing an amplitude of the breath signal at a point in time. The samples may not directly correspond to the amplitude of the breath waveform but should be understood as a digital representation of it. Similarly, the waveform may be encoded in any suitable manner such as an array of binary vectors or matrices.

Preferably, the breath waveform represents a single respiratory cycle. Obtaining the breath waveform may comprise splitting the capnogram into a plurality of capnogram sections, wherein each capnogram section represents a single breath waveform corresponding to the single respiratory cycle.

The single respiratory cycle is preferably a full recorded breath. That is, a breath recorded from exhalation to inhalation or vice versa, with complete recording of both exhalation and inhalation. The breath waveform/respiratory cycle may also correspond to a partial breath (e.g. where the recording of capnography data was started or stopped mid-breath). Transition points may still be determined as discussed below (and features can be extracted using those determined points) using the techniques of the present invention even when the breath waveform corresponds to a partial breath. However, depending on when the recording of capnography data was started or stopped, it may not be possible to determine as many transition points or extract as many features compared to a breath waveform corresponding to a full breath. Therefore, breath waveforms corresponding to partial breaths may be identified and accounted for (e.g., filtered out of the analysis). Having the breath waveform represent a single respiratory cycle reduces the amount of computation required while still providing the information required (e.g., the transition points) for the trained machine learning model to accurately generate an indicator of smoking history. This also facilitates further analysis such as average waveform generation, and the identification of anomalies in a capnogram.

Some machine learning or statistical inference models have been found to be particularly effective (e.g. provide a high level of accuracy) when used in the methods of the invention. Thus, it is preferred that the machine learning model comprises at least one of: logistic regression, a gradient boosting decision tree, a support-vector machine, ensemble methods such as AdaBoost, and a random forest. This is not an exhaustive list of models.

The one or more features may be extracted from the one or more breath waveforms in a variety of ways. For example, extracting one or more features from the one or more breath waveforms may comprise: normalising the duration of the one or more breath waveforms to generate a corresponding one or more normalised breath waveforms; generating an average breath waveform from the one or more normalised breath waveforms; and extracting the one or more features from the average breath waveform.

Preferably, the average breath waveform is generated from the plurality of normalised breath waveforms using a generalised additive model, GAM.

In this way, a single, smooth, average breath waveform is generated from which features can then be extracted. The extraction of features from this average breath waveform can be carried out instead of or in addition to the extraction of features from individual breath waveforms.

Optionally, the duration and/or the amplitude of the plurality of breath waveforms are normalised to generate the plurality of normalised breath waveforms. That is, the normalised breath waveforms have been normalised with respect to duration and/or amplitude.

Normalising the amplitude of the plurality of breath waveforms may comprise: extracting an end-tidal CO₂ value from each breath waveform; adjusting the amplitude of each of the plurality of breath waveforms such that each of the plurality of breath waveforms has the same end-tidal CO₂ value. The amplitude of the plurality of breath waveforms may be normalised based on various points of the individual breath waveforms such as the maximum value of the breath waveform or other transition points such as the alpha transition point. However, it has been found that normalising the waveforms based on the end-tidal CO₂ value consistently provides normalised waveforms which are well suited to use with the GAM to generate a smooth and accurate average breath waveform.

Extracting the end-tidal CO₂ value from each breath waveform may comprise, for each of the plurality of breath waveforms, determining: a beta transition point between the expiratory plateau and the inspiratory downstroke according to any of the techniques described below.

Extracting one or more features from the average breath waveform and/or from the one or more breath waveforms comprises: determining one or more transition points of the average breath waveform and/or the one or more breath waveforms; and extracting the one or more features using the one or more transition points; wherein the one or more transition points comprise one or more of: an alpha transition point between the expiratory upstroke and an expiratory plateau; a beta transition point between the expiratory plateau and the inspiratory downstroke; a gamma transition point between an inspiratory downstroke and an inspiratory baseline; and a delta transition point between an expiratory baseline and an expiratory upstroke.

Determining the transition points of the waveform enables accurate and consistent feature extraction to be performed on different segments of the breath waveform. In embodiments in which the indicator of smoking history is generated using the one or more extracted features as inputs to a trained machine learning model, this results in the trained machine learning model being able to more accurately generate an indicator of smoking history and for the model to generate this in a way which is explainable. It has been found that the alpha transition point, the beta transition point, the gamma transition point, and the delta transition point of a breath waveform are each particularly beneficial to the accuracy of generating an indicator of smoking history.

Capnograms and breath waveforms produced by different respiratory tracts can vary dramatically, as can capnograms and breath waveforms produced by the same respiratory tract at different times or under different conditions (e.g. when the respiratory tract is affected by a cardiorespiratory disease compared with a healthy respiratory tract). These variations have led to difficulties when attempting to accurately analyse a capnogram quickly and/or efficiently. Automating capnogram analysis has been particularly difficult due to the substantial degree of variation between breath waveforms and the wide range of factors (such as cardiorespiratory diseases, age, weight, time of day, medication use, smoking status, location, presence of other medical diseases etc.) which can contribute to these variations. However, determining the transition points of the waveform, as well as using these as the basis for feature extraction and application of the machine learning model, can be readily automated in a consistent manner to improve the efficiency of the model while still maintaining consistently high accuracy of the generated indicator.

As the transition points mark the transitions between different phases of the breath waveform (e.g. the alpha transition point is between the expiratory upstroke and expiratory plateau), determining any of these points also improves the interpretability of the method. That is, as well as generating an indicator of smoking history from the extracted features, the trained machine learning model can also indicate which phase(s) of the breath waveform contributed to the smoking indicator and to what extent.

As noted above, the breath waveform may be a digitally sampled signal representing a quantized amplitude signal which may be encoded as an array of floating point samples, each representing an amplitude of the breath signal at a point in time. The transition point may be the sample of the digital samples corresponding to the transition point of the waveform. The method may identify the individual sample or element in the encoding, an index of the sample, or the point in time corresponding to the sample. Each sample may generally correspond to a CO₂ value at a point in time.

Optionally, the waveform may be segmented into an expiratory phase and inspiratory phase based on the determined beta transition point.

Preferably, determining the plurality of transition points of a breath waveform, including of an average breath waveform, comprises determining a derivative of the breath waveform. The derivative of the breath waveform may be helpfully implemented in several steps of the method for a variety of reasons. Several of these implementations are discussed in detail below. For example, the derivative of the breath waveform may be used as a reference against the breath waveform in order to facilitate determining of the transition point(s). Preferably, the derivative of the breath waveform is a first order differential of the breath waveform, as the first order differential typically comprises less noise than higher order derivatives. However, higher order derivatives such as a second order differential may be more appropriate for determining the plurality of transition points depending on the breath waveform being examined. Determining the plurality of transition points may also comprise determining a plurality of derivatives of the breath waveform. For example, the first order differential of the breath waveform may be used to determine a given transition point, and the second order differential of the breath waveform used to determine a different transition point.

The derivative of the breath waveform, such as the first order differential, may also be used to identify, and optionally reject, anomalous breath waveforms, thereby preventing unnecessary processing being performed. In some implementations of the first or second aspect, the method further comprises comparing the derivative to a template; and, when the derivative is not consistent with the template, rejecting the breath waveform. The template may be a differential template or another type of template. Using the derivative to identify (and reject) anomalous breath waveforms may also comprise determining whether values of the derivative are within expected ranges.

Anomalous breath waveforms can also be identified, and optionally rejected, through other means. For example, by comparing values (e.g., at random or predetermined points) of the breath waveform itself or of the derivative to expected values or ranges. The template and these expected values are determined through empirical observations of typical physiology.

Preferably, determining the derivative of the breath waveform, such as the first order differential of the breath waveform, comprises applying a time-based smoothing filter, such as a Savitsky-Golay filter, to the breath waveform. For example, the first order differential of the breath waveform is determined in conjunction with application of a Savitsky-Golay filter. The time-based smoothing filter applies smoothing and so is particularly advantageous when used with noisy data. The Savitsky-Golay filter is also very generalisable, including the use of many window sizes and orders of polynomial fitting, and so has been found to be effective with a wide range of different breath waveforms. Other examples of smoothing filters include frequency filtering, e.g. using a wavelet or short-time Fourier filter, and moving average smoothing function.

Artefacts in breath waveforms such as hump artefacts have significant impacts on the shape of the waveform and its corresponding derivatives. In some cases, for example depending on the size and position of the artefact, this can lead to inconsistent feature extraction and less accurate generation of an indicator of smoking history, or less accurate training of the machine learning model. Accounting for any hump artefacts is of particular importance when automating the method for generating the indicator of smoking history (or the training method) in order to generate the indicator on a reasonable timescale. Hump artefacts can vary dramatically depending on the respiratory tract that produced the capnogram, however they are typically represented in a breath waveform by a sharp increase in the pCO₂ before the expiratory upstroke. The increased pCO₂ of the hump is less than that of the expiratory plateau and may remain at the increased hump level until the expiratory upstroke, may partially decrease into the expiratory upstroke, or may decrease and fully return to the pCO₂ levels of the expiratory baseline.

Therefore, it is preferred that determining the plurality of transition points comprises: identifying a hump artefact in the breath waveform and, when there is a hump artefact, accounting for the hump artefact during the determining of the one or more transition points.

The hump artefact may be accounted for through a variety of manners. For example by ignoring or removing the data associated with the hump artefact, in effect subtracting out the hump artefact, or adjusting the weighting applied to the data associated with the hump artefact when determining the one or more transition points.

Preferably, identifying the hump artefact comprises: performing peak detection to identify local minima of the breath waveform; identifying prominent minima from the local minima; identifying the maximum value of the breath waveform and/or determining the beta transition point; dividing the breath waveform into a first section not including a maximum value of the breath waveform, and a second section including the maximum value of the breath waveform and/or the beta transition point; when at least one prominent minima is identified, searching for hump artefact(s) in the first section of the breath waveform; and/or when no prominent minima are identified, using the derivative of the breath waveform to search for hump artefact(s) in the first section of the breath waveform. When the derivative of the breath waveform is a first order differential of the breath waveform, using the derivative of the breath waveform to search for/identify hump artefact(s) may comprise analysing the first order differential to identify an inflection point region of the first order differential, and comparing this inflection point region to a predetermined threshold.

The maximum value of the breath waveform refers to the maximum amplitude of the breath waveform. For example, the maximum pCO₂ value measured at a point in time during the breath waveform.

The breath waveform is preferably divided in the time dimension. That is, values up to a certain point in time (the point bounding the first and second sections) are in the first section and values after that point in time are in the second section.

The boundary between the first section and the second section may be the maximum value of the breath waveform.

Alternatively, the breath waveform may be divided such that the first section and second section are defined with respect to the beta transition point. That is, the breath waveform is divided into a first section not including the beta transition point and the second section includes the beta transition point.

It has been found that minima corresponding to hump artefacts typically have CO₂ values below a certain value. Therefore, to further reduce the computational cost, a hump artefact threshold may be implemented during the identifying of the hump artefact(s). For example, the hump artefact threshold may define a maximum CO₂ value where any minima with CO₂ values above the hump threshold are discarded or not considered during the identification of the hump artefact(s). Preferably, the hump artefact threshold is from 0.5 kPa to 4 kPa. Using a threshold within this range has been found to achieve an accurate level of hump artefact for most breath waveforms (e.g., by ignoring minima in the expiratory plateau). Most preferably, the hump artefact threshold is 2 kPa.

The first section and the second section may alternatively be defined as non-overlapping sections, where the second section includes the maximum value of the breath waveform and/or the beta transition point.

The beta transition point may be determined through various means. It has been found that a reliable and efficient method (particularly during automation) of determining the beta transition point comprises: performing peak detection to find local maxima of the breath waveform; identifying prominent maxima from the local maxima; when only a single prominent maximum is identified, determining this as a beta transition point between the expiratory plateau and the inspiratory downstroke; and, when a plurality of prominent maxima are identified, determining the most prominent maximum and defining this as the beta transition point.

It has been found through extensive data analysis that the pCO₂ value of the beta transition point typically falls within or below a range of values. Therefore, a predetermined threshold (i.e., a maxima threshold) may be defined to save further processing resources. In practice, this can be implemented by ignoring maxima with (pCO₂) values below the maxima threshold (e.g., by removing these values or setting them equal to 0 during the peak detection or identification of prominent maxima. This reduces the processing resources used by the method and reduces noise from values before the maxima threshold. For example, the maxima threshold may be set to a value from 0.04 kPa to 5 kPa. These lower and upper values for the threshold account for background pressure while reliably and accurately determining the beta transition point such that accurate and useful features can be extracted based on the determined beta transition point. Preferably, the maxima threshold may be set from 1.5 kPa and 2.5 kPa. Most preferably, the maxima threshold is 2 kPa. The maxima threshold may be predetermined or, in some implementations, the maxima threshold may be determined and/or adjusted by a machine learning model as it continues to determine beta transition points of an increasing quantity of breath waveforms. This means the machine learning model can further optimise the resource usage of the methods.

When no prominent maxima are identified, the breath waveform may be rejected as this is normally due to the breath waveform being an anomaly where extracted features are less likely to be accurate or may be unreliably interpreted by a machine learning model (that is, during indicator generation or training). Rejecting the breath waveform prevents these outcomes at an early stage without any further unnecessary processing.

Preferably, determining the transition points comprises determining the delta transition point, and determining the delta transition point comprises: determining the first point in time at which a first order differential of the breath waveform is above a delta threshold; and defining the first point as the delta transition point.

Similarly to the maxima threshold, the delta threshold may be a predetermined threshold, or may be determined and/or adjusted by the (trained or untrained) machine learning model. Optionally, the delta threshold is configured relative to the maximum magnitude value of the first order differential of the breath waveform.

For example, the delta threshold may be from 5% to 80% of the maximum magnitude point of the first order differential. Preferably, the delta threshold is from 5 to 15% of the maximum magnitude point of the first order differential. More preferably, the delta threshold is 10% of the maximum magnitude point of the first order differential. Using these ranges for the delta threshold, and the specific value of 10%, have been found to accurately determine the delta transition point such that accurate and useful features can be extracted based on the determined delta transition point.

Preferably, determining the transition points comprises determining the gamma transition point, and determining the gamma transition point comprises: identifying the minimum value of a first order differential of the breath waveform; and, defining the gamma transition point as the first point in time after the minimum value at which the first order differential of the breath waveform is higher than a gamma threshold.

The first point in time refers to substantially the first point in time, and may be near the first point in time or, for example, a data point closely corresponding with the first point in time.

Similarly to the previously discussed thresholds, the gamma threshold may be a predetermined threshold, or may be determined and/or adjusted by the (trained or untrained) machine learning model. Optionally, the gamma threshold is configured relative to the maximum magnitude value of the first order differential of the breath waveform. For example, the gamma threshold may be from 0 to the negative of 50% of the maximum magnitude point of the first order differential. Preferably, the gamma threshold is from the negative of 2% and the negative of 10% of the maximum magnitude point of the first order differential. More preferably, the delta threshold is the negative of 5% of the maximum magnitude point of the first order differential. Using these ranges for the gamma threshold, and the specific value of −5%, have been found to accurately determine the gamma transition point such that accurate and useful features can be extracted based on the determined gamma transition point.

Preferably, determining the transition points comprises: determining the alpha transition point, wherein determining the alpha transition point comprises: identifying the maximum value of a first order differential of the breath waveform; identifying the maximum value of the breath waveform and/or determining the beta transition point; and, defining the alpha transition point as the first point in time, between the maximum value of the first order differential and the maximum value of the breath waveform and/or the beta transition point, at which the first order differential of the breath waveform is less than an alpha threshold.

It will be apparent that the alpha transition point may be determined using the maximum value of the breath waveform or using the beta transition point. These two methods may be performed individually (i.e., only one is performed in order to determine the alpha transition point while using less processing resources) or both may be performed to verify the result of the other. The beta transition point only needs to be determined at this stage if this has not already been achieved, otherwise the previously determined beta transition point may be used, or the alpha transition point may be determined without using the beta transition point. Determining the alpha transition point (and/or identifying hump artefact(s)) using the beta transition point has been found to be a more reliable method than using the maximum value of the breath waveform and is suitable for a wider range of breath waveform shapes. However, the techniques using the maximum value of the breath waveform are reliably accurate with lower computational costs associated.

Similarly to the previously discussed thresholds, the alpha threshold may be a predetermined threshold, or may be determined and/or adjusted by the (trained or untrained) machine learning model. Optionally, the alpha threshold is configured relative to the maximum magnitude value of the first order differential of the breath waveform. For example, the alpha threshold may be from 0 to 80% of the maximum magnitude point of the first order differential. Preferably, the alpha threshold may be from 10% and 20% of the maximum magnitude point of the first order differential. More preferably, the alpha threshold is 15% of the maximum magnitude point of the first order differential. Using these ranges for the alpha threshold, and the specific value of 15%, have been found to accurately determine the alpha transition point such that accurate and useful features can be extracted based on the determined alpha transition point.

In some implementations of the method, the alpha threshold may be adjusted to reduce the influence of noise on the determining of the alpha transition point, and to improve the processing resource efficiency of the method. Therefore, determining the alpha transition point may further comprise: when no point between the maximum value of the first order differential of the breath waveform and maximum value of the breath waveform is less than the alpha threshold, increasing the alpha threshold. The alpha threshold may be increased by a predetermined amount. For example, the alpha threshold may be increased by 5% of the maximum magnitude point of the first order differential of the breath waveform.

After the alpha threshold has been increased, the values of the first order differential between (i.e., between points in time corresponding to) the maximum value of the first order differential and the maximum value of the breath waveform and/or the beta transition point can be compared with the increased alpha threshold in order to determine and define the alpha transition point. When no point meets this definition, the alpha threshold can be increased again and this process performed iteratively until the alpha transition point has been determined. The amount that the alpha threshold is increased may vary in different iterations.

In order to reduce unnecessary processing resources used during the method, an upper limit can be placed on the number of iterations performed (and therefore the number of times the alpha threshold is increased) and/or the value of the alpha threshold itself, and when one of these limits is reached the breath waveform is rejected.

The alpha transition point may also be determined using further alternative methods. One such alternative method of determining the alpha transition point comprises: calculating a line between the delta transition point and the maximum value of the breath waveform, or calculating a line between the delta transition point and the beta transition point; and, defining the alpha transition point based on the distance between the breath waveform and the calculated line.

For example, the alpha transition point may be defined as the point of the breath waveform, between the delta transition point and the maximum value of the breath waveform (or the beta transition point), that is furthest from the line calculated between the delta transition point and the maximum value of the breath waveform (or between the delta transition point and the beta transition point), where the distance between the breath waveform and the calculated line is measured using another line that is perpendicular to the (first) calculated line (between the delta transition point and the maximum value of the breath waveform or the beta transition point).

This may be implemented as an alternative method of determining the alpha transition point to that using the alpha threshold or used as a supplementary method of determining the alpha transition point to corroborate another method.

In an alternative implementation to the above algorithmic approach, determining the transition points may comprise: applying a trained machine learning model to a set of discrete samples of the breath waveform, the breath waveform representing a whole breath, wherein the machine learning model is configured to classify each sample into one of a plurality of output classes, each class representing a region of the breath waveform, and wherein the machine learning model is trained by: obtaining a label associated with each discrete sample of a plurality of breath waveforms, each breath waveform being represented by a set of samples representing a whole breath and each label indicating which of a plurality of output classes that sample corresponds to; and, training the machine learning model on the labels and the samples to learn to classify a sample of a set of samples representing a whole breath into a class of the plurality of output classes. This approach may provide consistency for feature engineering but may be difficult to consider all types of breaths and accuracy will scale with the volume of labelled input data.

A large variety of features of the breath waveform may be extracted using the determined transition points. For example, the extracted features may include at least one of: the time and/or CO₂ pressure at transition points or any other point of the breath waveform, the end-tidal CO₂, angles between linear fits either side of a transition point, duration of phases between transition points, the ratio of the angle between linear fits and the duration of the phase(s), the minimum, maximum, average, median and/or total CO₂ pressure during the phases between transition points, the respiratory rate, coefficients of lines fitted to the breath waveform using the transition point(s).

Further examples include: hyperbolic tangent fits, a delta angle, an alpha angle, a beta angle, a gamma angle, a delta angle start, a delta angle finish, an alpha angle start, an alpha angle finish, a beta angle start, a beta angle finish, a gamma angle start, a gamma angle finish and the time and CO₂ values at these points, the minimum CO₂ in the inspiratory baseline of the breath waveform, the minimum CO₂ in the expiratory upstroke of the breath waveform, the ratio of the alpha angle to the duration of the expiratory phase, the CO₂ at the centre of the gamma angle, the coefficients of a quadratic fit to the expiratory plateau, the time difference between any of the determined transition points, breath patterns and measures of disorder such as entropy.

Features relating to breath patterns can be detected by assessing periodicity of breath waveforms (e.g. using autocorrelation or frequency-domain identifiers) and comparing variability in breaths.

The delta angle start refers to the capnogram values at the delta angle start, for example the time and/or pCO₂ values at the delta angle start. This is the same for the start and finish points of the alpha, beta and gamma angles.

Extracting features of a breath waveform using the one or more transition points may comprise determining an angle of each of the one or more transition points. The determination of an angle of each of the one or more transition points may comprise, for each of the one or more transition points: fitting a first linear function and a second linear function to the adjacent phases on either side of the transition point, and measuring the angle between the first and second linear functions. For example, if the angle of the alpha transition point is being determined then fitting the first/second linear function to the adjacent phases on either side of the alpha transition point means fitting the first linear function to the expiratory upstroke and the second linear function to the expiratory plateau.

Extracting features of the breath waveform using the one or more transition points may also comprise fitting a quadratic function to the expiratory plateau, and determining one or more coefficients of the quadratic function. This provides an indication of the “flatness” of the expiratory plateau which can be used to help determine characteristics of the respiratory tract associated with the breath waveform. Preferably, the quadratic function is fitted across the full length of the expiratory plateau from the alpha transition point to the beta transition point.

As a further example, extracting features of the breath waveform using the one or more transition points may comprise fitting a hyperbolic tangent function to the expiratory upstroke and/or the inspiratory downstroke, and determining one or more coefficients of the hyperbolic tangent functions. These are coefficients such as the width and horizontal displacement parameters of the fitted hyperbolic tangent functions. Preferably, the hyperbolic tangent functions are fitted across the full length of the expiratory upstroke (i.e., between the delta and alpha transition points) and the full length of the inspiratory downstroke (i.e., between the beta and gamma transition points).

Preferably, the method further comprises obtaining a capnogram from a user, wherein the capnogram comprises a breath waveform.

In the above described embodiments, the method of generating the indicator of smoking history using the trained model and the method of training may each be performed remotely from the user from which the capnogram was produced, at a local computer or at a remote computer. Steps of the method may be performed entirely by a local processing unit, remote processing unit such as in the cloud, or split between local and remote processing units.

According to a third aspect, there is provided an apparatus configured to perform a method according to any of the embodiments of the first and second aspects of the invention.

According to a fourth aspect, there is provided a computer readable medium comprising instructions which, when executed by a processor, cause the processor to perform a method according to any the embodiments of the first and second aspects of the invention.

### BRIEF DESCRIPTION OF DRAWINGS

Embodiments of the invention are described below, by way of example only, with reference to the accompanying drawings, in which:

FIG. 1A shows a breath waveform from a healthy patient;

FIG. 1B shows a breath waveform from a patient with a cardiorespiratory disease;

FIG. 1C shows a breath waveform including an artefact;

FIG. 2 shows examples of breath waveforms produced by users having different smoking histories;

FIG. 3 shows an example capnogram;

FIG. 4 shows an idealised breath waveform;

FIG. 5 shows a method for generating an indicator of smoking history according to examples of the present disclosure;

FIGS. 6A to 6D show different breath waveforms together with the first order differential of the breath waveforms;

FIGS. 7A to 7C show a schematic process flow for identifying hump artefacts, transition points, and features of the breath waveform;

FIG. 8 shows a method for training a machine learning model to learn an indicator of smoking history according to examples of the present disclosure;

FIG. 9 shows a schematic of an example of the present disclosure applied to two components;

FIGS. 10A and 10B illustrate the three components of FIG. 9 in schematic detail; and

FIGS. 11A and 11B respectively illustrate a regression plot of CO₂ alpha-angle feature vs pack years and average CO₂ waveforms across subjects with <15 pack years vs >30 pack years smoking history.

### DETAILED DESCRIPTION

Specific embodiments of the invention are discussed in detail below, relating to generating an indicator of smoking history from one or more capnograms produced from a user and to training a machine learning model to learn an indicator of smoking history.

Many of the following embodiments are described with reference to using a trained machine learning model to generate an indicator of smoking history from one or more capnograms produced from a user. However, the present disclosure is not limited to using a machine learning model to generate this indicator.

Recent hardware innovations, such as those described in WO2017174983 and WO201915800, the contents of which are each incorporated herein by reference, have led to it being possible to measure CO₂ concentration reliably and non-invasively at the mouth, closer to the airway and with a high time resolution. This has allowed for greater flexibility in pulmonary health assessment, providing more insight than would previously be recognised.

In particular, an objective of the present disclosure is to use one or more capnograms produced from a user to determine the user's smoking history. This smoking history can be based on indicators such as pack-years and lung age, although the present disclosure is not limited to these indicators.

Other objectives are to provide explainability of the generated indicators so that clinicians are able to understand what contributed to the indicator and to provide a computationally efficient process suitable to be deployed at the edge, for example so that an implementation stage can implement data processing, feature extraction, and indicator generation using a trained model at the clinician's site without cloud communication. Optionally, for example, the indicator generation methodology may be implemented on chip, i.e. on a handset, or tasks may be divided between the handset and a local computer.

Methodologies proposed herein have particular beneficial use in accurately generating a metric that summarises a user's smoking history, such as number of years of smoking, current vs ex vs never smoker, years since quit smoking, age first smoked, grams of tobacco smoked (if smoking a pipe or roll ups for example), among others. As used herein, “smoking” is not limited to tobacco smoking and includes tobacco smoking, cannabis use, vaping, any combination of these three, or any other forms of smoking.

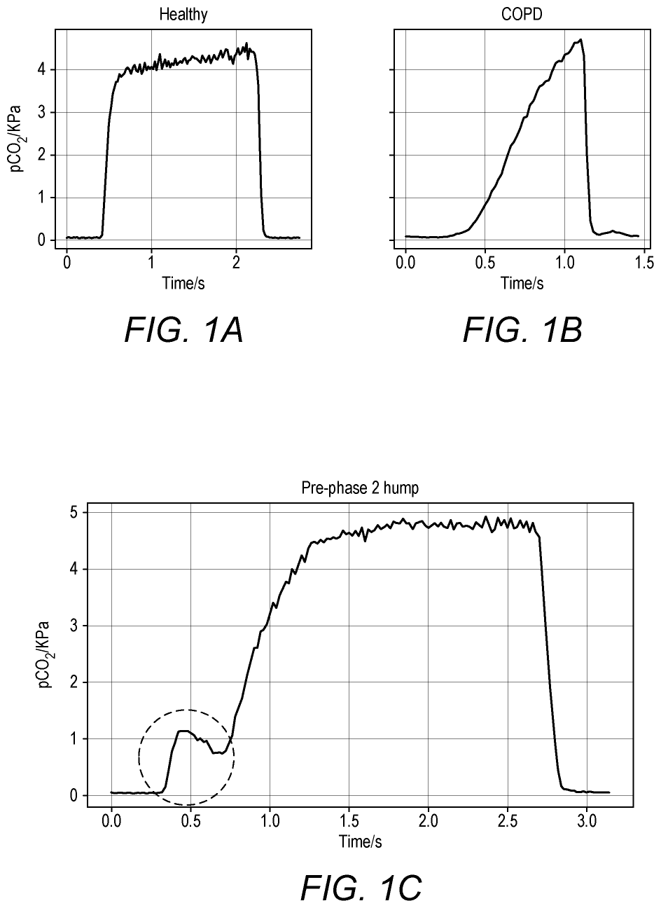

FIGS. 1A-1C show three examples of different breath waveforms, where the breath waveforms plot the partial pressure of carbon dioxide (pCO₂) as a function of time as a patient performs a single breath by exhaling and then inhaling. The terms partial pressure of carbon dioxide, concentration of carbon dioxide, and carbon dioxide are used interchangeably throughout the description unless otherwise specified. FIG. 1A shows an example breath waveform produced by the breathing of a healthy patient, FIG. 1B shows an example breath waveform produced by the breathing of a patient with a cardiorespiratory disease (in this case COPD), and FIG. 1C shows an example breath waveform of a patient whose breathing produces a hump-like breath artefact in the waveform at the outset of the patient's exhalation.

Deoxygenated but CO₂ rich blood arrives through the pulmonary vasculature at the alveoli where gaseous exchange takes place. Perfusion exchanges O₂ in the ventilated air with carbon dioxide in the blood to facilitate respiration. The relaxation of the diaphragm then decreases the volume of the thoracic cavity during exhalation, thereby increasing pressure, and forcing CO₂ out through the bronchioles and into the environment. Although many implementations use volumetric capnometry, this disclosure focuses on time capnometry, which aims to measure the partial pressure of carbon dioxide (pCO₂) over time. It will be understood that aspects of the invention may also be applicable to volumetric capnometry. In an ideal gas mixture, the relationship of the total pressure to the individual gas partial pressures is given by

$${p}_{tot} = {\sum }_{i} {p}_{i}$$

where pᵢ is the partial pressure of gas i, which can be expressed as

$${p}_{i} = {p}_{t o t} \frac{{n}_{i}}{{n}_{tot}}$$

with nᵢ as the number of molecules of gas i in any macroscopic volume and nₜₒₜ the number of molecules in the entire gas mixture in the same volume. At sea level, atmospheric pressure is pₐₜₘ=1 atm≈101 KPa, with background pcO₂≈0.0417 KPa, dependent on the environment.

Typically, as a patient exhales, the concentration of CO₂ at the mouth increases sharply, from baseline, and typically reaches a plateau as diffusion begins to compensate. The inhalation then brings atmospheric CO₂ back towards the mouth, decreasing concentration back to baseline. The end of the plateau phase is referred to as the end-tidal CO₂ (ETCO₂), and is an important biomarker for anesthetists.

Patients with an obstructive cardiorespiratory disease typically exhibit a more ‘shark-fin’ shape to their capnographic breath waveforms, as highlighted in FIG. 1B. During exhalation, damaged and/or inflamed alveoli and/or airways may limit perfusion, may degrade the elasticity of the alveoli walls, and may alter the physiology of the airways, altering the force at which gases can be exhaled, typically leading to a more gradual expiratory upstroke, a larger delta angle, and a steeper expiratory plateau.

It is clear from FIGS. 1A to 1C that the breath waveforms vary depending on the condition of a patient and so can be examined to predict information about the patient.

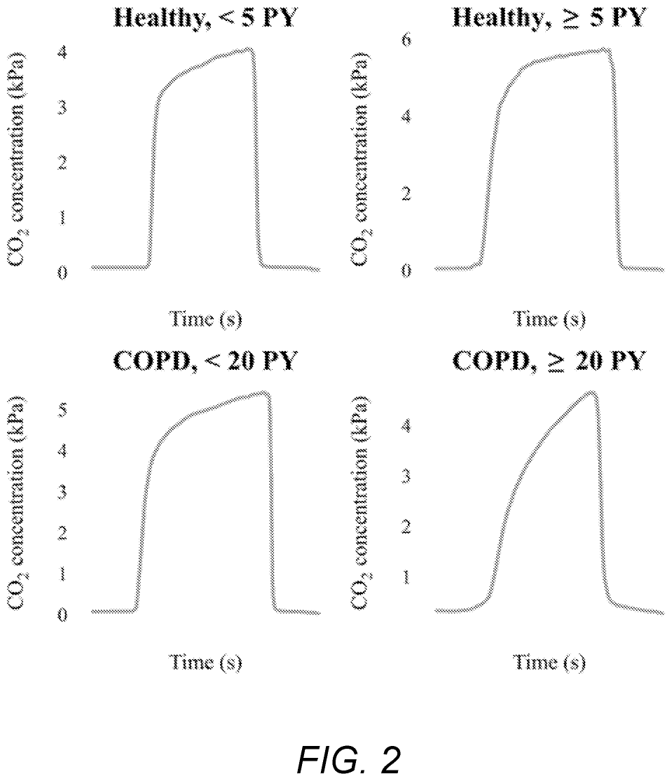

The breath waveforms produced by a user will also vary depending on the smoking history of a user, as shown in FIG. 2. This figure shows four examples of breath waveforms produced by users with different health conditions and smoking history. The smoking history is quantified in these figures using the metric of pack years, a measure of the total number of cigarettes smoked during their lifetime, where 1 pack year is defined as having smoked a pack of 20 cigarettes every day for a year, i.e. 7,305 cigarettes.

As an example, for a healthy user, the shoulder between the inspiratory upstroke and the expiratory plateau (the a transition point) is more defined when the number of pack years are lower than 5 compared with a healthy user having more than 5 pack years.

The difference is even more pronounced for a user exhibiting COPD, with a user having greater than 20 pack years exhibiting significant rounding of the alpha transition point leading to a ‘shark-fin’ shape. In comparison a capnogram produced from a user exhibiting COPD but having fewer than 20 pack years, while still exhibiting more rounding of the alpha transition point than a capnogram produced from healthy user having fewer than 5 pack years, exhibits far less rounding of the alpha transition point.

The alpha transition point is not the only feature which can be used to determine the pack years of a user, and other metrics may also be used as indicators of smoking history. Indeed, breath waveforms vary in a large variety of specific manners and so many different features may be extracted from a waveform and examined in order to generate an indicator of smoking history. These waveform variations and features will be discussed in more detail below.

As mentioned, recent innovations have led to the possibility of taking reliable recordings of pCO₂ directly from the mouth, with high sensitivity using a dedicated handset. It will be understood however that innovations disclosed herein are not limited to data derived from such a handset but description is provided for context. The patient performs normal tidal breathing through the mouthpiece for a period of ≈75 seconds resulting in a non-invasive and effort-independent experience.

Carbon dioxide strongly absorbs electromagnetic radiation of wavelength 4.3 μm or 15 μm, where the energy causes its intramolecular bonds to vibrate before being re-emitted at different wavelengths in different directions. An emitter in the handset emits photons of either of these wavelengths through the air pathway, where the unabsorbed photons are detected by a sensor whose output is dependent on the partial pressure of carbon dioxide within the air pathway's volume. The handset differs from conventional capnometers in that it does not have a reference channel; instead, the device self-calibrates on power up in reference to the background CO₂ level at the time of use. The sensor also has a fast response time because of a combination of the speed of respired breath through the sampling volume and because the sampling volume is adjacent to the mouth and so is unaffected by differential velocities due to wall drag when sampling distally from the mouth (as is the case with alternative technologies).

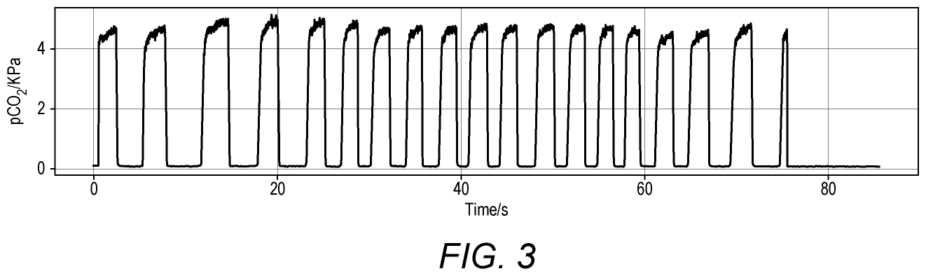

The resulting output is sampled at 10 kHz, and reported at 50 Hz, which is much higher than any other capnometry monitor on the market, whereafter the anonymous data is automatically, and securely uploaded via mobile network to a cloud platform. Here, it is run through a processing pipeline, as set out below in accordance with aspects of the disclosure, and made available for subsequent analysis. An example of a full raw breath record is displayed in FIG. 3.

FIG. 3 shows an example of a capnogram illustrating a tidal breath record, also plotting the partial pressure of carbon dioxide (pCO₂) as a function of time as a patient breathes. It will be apparent from comparing FIGS. 1A-1C and FIG. 2 with FIG. 3 that the capnogram of FIG. 3 illustrates over a dozen full breaths, along with partial breaths at the beginning and the end of the capnogram. The number of breath waveforms in a capnogram will vary depending on the length of time a patient's breathing is measured.

Input to the process which is the object of the present disclosure may accordingly be a set of digitised samples at a particular sampling rate, with each digital sample representing an amplitude of the pCO₂ at a particular point in time. The samples may be represented as a set of vectors, each with a corresponding time index. This time-series data represents the input to the methodology.

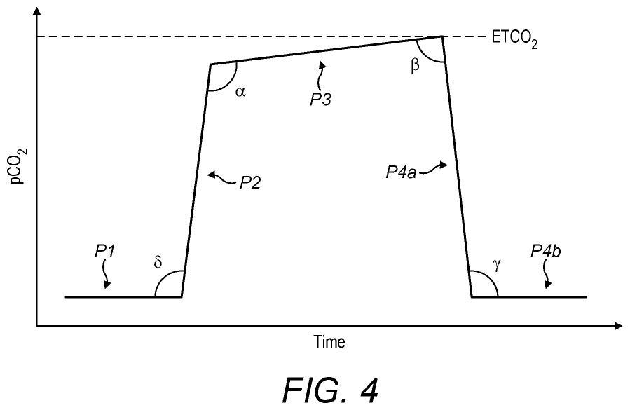

According to the present disclosure, a classifier has been developed that utilises several key geometric aspects identified common among breath waveforms which can be determined in order to facilitate feature extraction and the generation of indicators of smoking history from the respective biomarkers. These geometric aspects, or biomarkers, have hitherto not been capable of reliable and accurate identification by a computer, as will be explained in more detail below. FIG. 4 shows an idealised breath waveform with several of these geometric features highlighted.

The illustrated idealised waveform may be segmented into five linear segments or phases, from left to right as time progresses these are an expiratory baseline P1, an expiratory upstroke P2, an expiratory plateau P3, an inspiratory downstroke P4a, and an inspiratory baseline P4b. The expiratory baseline P1 is also referred to as phase 1, the expiratory upstroke P2 is also referred to as phase 2, the expiratory plateau P3 is also referred to as phase 3, the inspiratory downstroke P4a is also referred to as phase 4a, and the inspiratory baseline P4b is also referred to as phase 4b. Typically, as a patient exhales, the concentration of CO₂ at the mouth increases sharply, from the expiratory baseline P1, and reaches a plateau (the expiratory plateau P3) as diffusion begins to compensate for the increased CO₂ concentration. The inhalation then brings atmospheric CO₂ back towards the mouth of the patient, decreasing concentration back to baseline (the inspiratory baseline P4b). It will be apparent from the capnogram of FIG. 3 that the inspiratory baseline P4b of a first breath waveform may extend into or be the expiratory baseline P1 of a second breath waveform, and vice versa. The expiratory baseline P1 and inspiratory baseline P4b are so named in the context of a single breath cycle, i.e. a single waveform representing one breath cycle, to assist explaining individual breath waveform analysis.

As will be discussed below, determining the location of at least one of the transition points between these five segments allows for reliable and efficient feature extraction of the breath waveform but doing so is not straightforward. The delta transition point δ is the point of the breath waveform bounding the expiratory baseline P1 and the expiratory upstroke P2. The alpha transition point α is the point of the breath waveform bounding the expiratory upstroke P2 and the expiratory plateau P3. The beta transition point β is the point of the breath waveform bounding the expiratory plateau P3 and the inspiratory downstroke P4a. The gamma transition point γ is the point of the breath waveform bounding the inspiratory downstroke P4a and the inspiratory baseline.

While the breath waveform in FIG. 4 is idealised with linear segments, this is not how breath waveforms appear in reality. Artefacts in a breath waveform such as the pre-expiratory upstroke P2 hump shown in FIG. 1C are common in capnograms and increase the difficulty of analysis (e.g. determining the location of transition point(s)) and feature extraction. Such artefacts are particularly problematic when automating the process or using a machine learning model to facilitate the generation of indicators of smoking history. Many other types of artefacts are also found in breath waveforms and are advantageously also be accounted for to ensure that a method for generating indicators of smoking history is robust against as many types of artefacts as possible.

Noise such as that in the measured pCO₂ signal also makes it more difficult to accurately determine the transition points, as does the general shape of the waveform. For example, it will be appreciated that the alpha transition point α in FIG. 1A's “square wave” waveform is more clearly defined and so easier to determine accurately than the same alpha transition point α in FIG. 1B's “shark fin” waveform.

Each of these issues (e.g. artefacts and noise) may become more apparent in capnograms produced with higher measurement frequencies. However, if the issues are correctly accounted for when determining transition point(s) and extracting feature(s) from a breath waveform, then the extracted features are more accurate and so too are the resulting indicators of smoking history.

Early implementations of computer-assisted methods consisted of a manual initial phase segmentation step that identifies each of the phases outlined in FIG. 4 through calculation of five features, including the maximum and minimum CO₂ values and the stroke gradients. A second step aimed to derive a template waveform by averaging the derived features between breaths before a third makes a comparison of subsequent breaths with this template. This enabled rejection of anomalous breaths with strange artefacts, the reason for which could be inferred by comparison of features to categories of breaths with known deformities. In similar approaches breaths were separated by pinpointing the positive and negative gradients, before a template waveform of CO₂ values was constructed from the average and outliers identified.

Rule-based methods have also been proposed, defining criteria on the absolute changes in CO₂ concentration as a way to extract phases for breath separation and further rules including the duration of breath were established for breath rejection. Other methods identified breath initiation and completion by finding inflection points in sliding windows where pCO2<16 KPa and rejected breaths based on pre-established extremes of physiological possibility. Another pre-processing approach includes fitting models to breaths. For example, segmenting breaths into phases by fitting a series of piecewise linear lines before splitting into individual breaths by application of logic to valleys. An analogous technique has been applied to identify segments by linearly modelling data in a sliding window of length 3 and rules applied to a plot of this slope fit over time.

Machine learning has also been used for breath segmentation where artificial neural networks (ANN) ingested the raw time series and output the start and ends of breaths. Similar solutions used an ANN on eight features derived from the waveform for poor-breath rejection.

These capnographs were mostly obtained from sedated patients, at low sampling rates, leading to greater uniformity in capnographic presentation.

None of these solutions provide an accurate, reliable technique for processing a high frequency noisy signal with artefacts leading to explainable and computationally efficient processing and indicator generation.

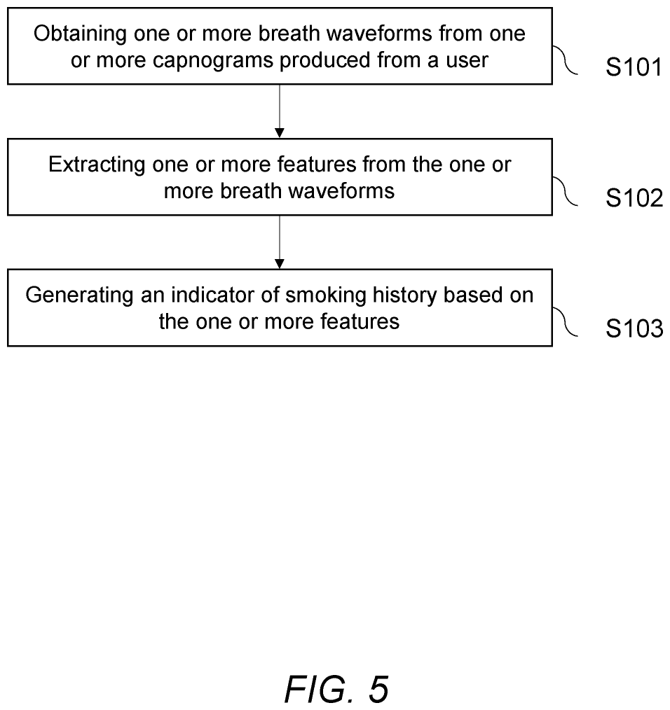

FIG. 5 shows a method for generating an indicator of smoking history according to examples of the present disclosure. Known methods are not able to accommodate the variety of artefacts which may influence the automated detection of the transition points.

In a first step, the method obtains one or more breath waveforms from one or more capnograms produced from a user (S101). This may be in the form of the digitised samples discussed above. From these breath waveforms, the method extracts one or more features (S102). As identified below, any number of features may be extracted from which the classifier is able to learn to predict a result from an unseen set of capnogram data. In an implementation phase, an indicator of the user's smoking history is generated based on the one or more features (S103). This may be achieved by applying a trained machine learning model to the extracted features.

In some embodiments, a set of transition points may be determined and used to extract the one or more features. In examples, these are identified by analysing a first order differential of the waveform and maxima and minima of the signal. Preferably the first order differential is calculated using a time-based smoothing filter in conjunction with a first order derivative. Local minima and maxima of the signal may be caused by breathing artefacts or physiological artefacts and may interfere with the detection of the transition points. Output of this step may therefore be a set of start and end points of each of the phases, i.e. an index of the relevant sample for example, and optionally a first order differential and values at each of the identified points.

It will be understood that in typical machine learning processes there may be three stages, training, validation and test. Each of these may be applied by different entities from features extracted from respective data sets using the above methodology.

In a general form, a supervised machine learning model takes input data, X∈R^{m×n} of m examples and n features sampled from χ, alongside paired output data, Y∈R^{m×k} sampled from γ, as an m×k matrix of the corresponding m examples and k outputs. The objective is to use an algorithm, A, that uses this data to create a function, ƒ∈γ^{χ}, that can take any unseen x∈χ and output a prediction y∈γ:

$$A : ( χ \times γ ) \to ( f : 𝒳 \to γ ) .$$

In this problem statement, χ is the set of featurised capnograms, and γ are the labels that specify an indicator of smoking history associated with the user from which a capnogram was produced. A disease label may also be used, and a distinction may be made between healthy, COPD, and non-healthy but non-COPD, as patients without COPD could have other diseases that affect the capnography waveform. This disease label could be used as an input into the trained machine learning model, or could be used as the basis for training different machine learning models associated with different disease labels and used at inference to select the appropriate trained machine learning model to generate an indicator of a smoking history of a user based on the presence or absence of a disease.

In step S101, one or more breath waveforms are obtained from one or more capnograms. When a capnogram includes a plurality of breaths (these may be full breaths and partial breaths) as in the example of FIG. 3, then obtaining a breath waveform includes splitting the capnogram into sections. When splitting the capnogram into sections, each capnogram section should preferably consist of a single breath waveform corresponding to a single respiratory cycle (i.e. a full breath or a partial breath if sampling of capnography data was started or stopped mid-breath).

One of the particular benefits of the present methodology is the ability to perform indicator generation accurately and reliably based on a single waveform. Preferably pre-processing steps such as de-noising and breath separation may take place but these are not essential and not described. A further pre-processing step to form a stylised single waveform is a further example of the disclosure and described below.

As noted above, one or more transition points of the waveform may be determined. In particular, the determined transition points include one or more of the delta transition point δ, the gamma transition point γ, and the alpha transition point α.

These transition points may be determined through a number of methods. Due to its high accuracy and compatibility with automation and machine learning, a preferred method includes determining a first order differential of the breath waveform. It has been found that the first order differential of the breath waveform may be used as a reference against the breath waveform itself to determine several of the transition points.

Determining the first order differential of the waveform also enables the breath to be screened for viability to save computing resources. For example, the first order differential can be compared to a differential template and, when the first order differential of the breath waveform is not consistent with that template, the breath waveform will be rejected and analysis of the waveform will cease. The nature of the template and level of inconsistency permitted before the rejection threshold can vary depending on the details of the waveform analysis; for example, which transition points are to be identified and which features of the breath waveform are intended to be extracted.

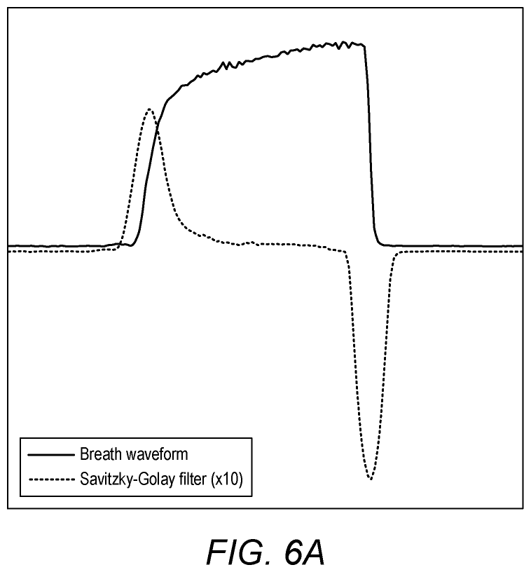

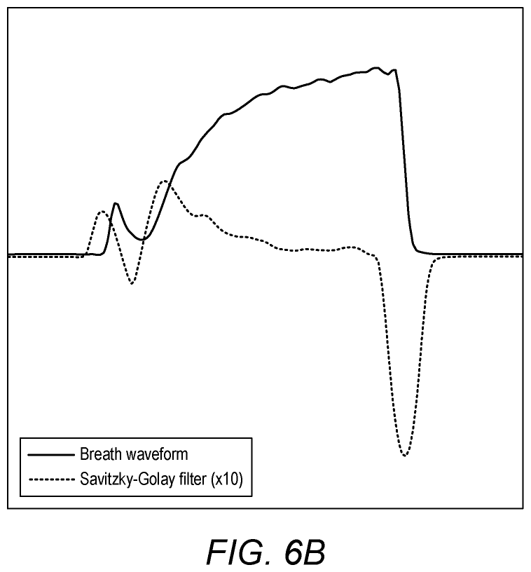

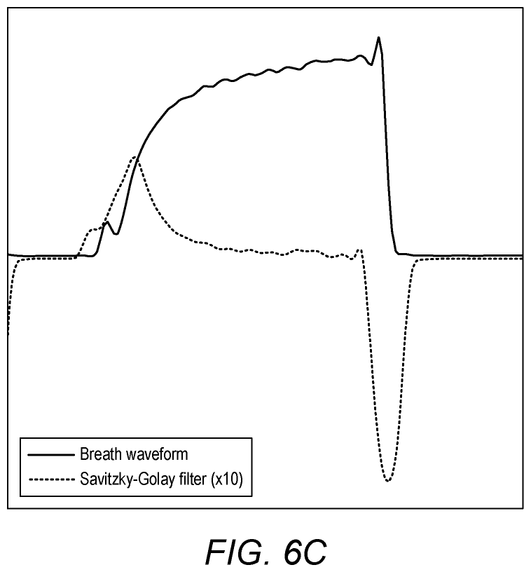

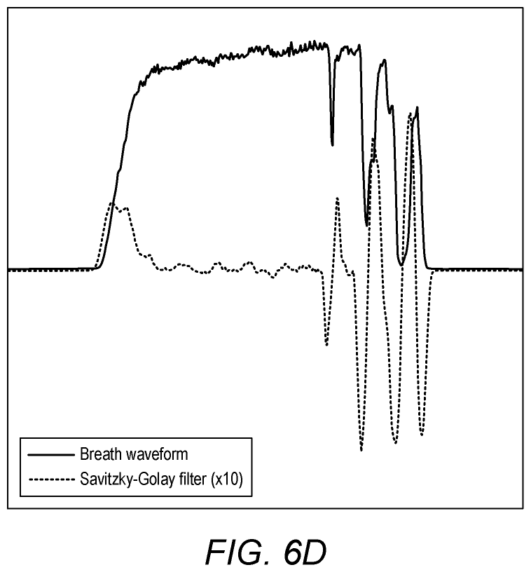

FIGS. 6A to 6D show several examples of breath waveforms overlayed by their first order differential, where the first order differential was determined in conjunction with a Savitsky-Golay (SG) filter applied to the breath waveform. The scales of the SG filter first order differential and breath waveform have been altered in these figures to facilitate easier comparison of the SG filter and the breath waveform. As will be apparent from FIGS. 6A to 6D, and in particular FIG. 6A when compared with the idealised waveform of FIG. 4, the peaks of the first order differential may substantially correspond, or be otherwise adjacent to, the transition points of the breath waveform and so may be used as part of determining the transition points.

It will be apparent from FIGS. 6B, 6C, and 6D that artefacts in the breath waveform have a significant effect on the shape of the waveform and the first order differential. For example, the pre-expiratory upstroke P2 hump artefact shown in FIGS. 1C, 6B, and 6C. These artefacts should preferably be identified and accounted for during waveform analysis (e.g., during determining of transition points and/or feature extraction). This is particularly beneficial when automating the step of determining the transition points.

Preferably, when a hump artefact is identified in a breath waveform it is accounted for during further analysis of the breath waveform. For example, if a hump artefact produces the maximum magnitude points on the first order differential, rather than this being caused by the expiratory upstroke P2 or the inspiratory downstroke P4a, the first order differential can account for the hump artefact by being normalised with respect to a peak caused by the upstroke P2 or the downstroke P4a instead of the hump artefact. Various thresholds used in determining transition point(s) (discussed below) may then be defined based on the re-normalised first order differential to avoid influence from the hump artefact. Alternatively, the hump artefact may be accounted for by ignoring the artefact, for example by ignoring or removing the data associated with the hump artefact (in effect, subtracting the hump out), or adjusting the weighting applied to the data associated with the hump artefact when determining the one or more transition points.

Artefact identification may be performed through several methods. Due to its high accuracy and compatibility with automation and machine learning, a preferred method includes performing peak detection operations on the breath waveform, and optionally also determining and/or using the first order differential of the breath waveform.

As part of the artefact identification, peak detection is performed to identify local minima of the breath waveform. Prominent minima may then be identified from the local minima. Identifying the prominent minima is particularly useful when the breath waveform is a noisy signal. When one or more prominent minima are found, the local area of the waveform is examined to determine whether there is a hump artefact and, when present, identify the hump artefact. For example, it will be apparent from FIG. 1C that there is a prominent local minima before the expiratory upstroke P2 and after a hump artefact, searching for local maxima near this prominent minimum will identify the pre-expiratory upstroke P2 hump artefact.

When no prominent minima are identified (e.g., due to a noisy breath waveform), the first order differential of the breath waveform may be used to search for hump artefacts. As shown from comparison of FIG. 6A with FIGS. 6B and 6C, the first order differential (in this case, an S-G filter) is significantly affected by the presence of a pre-expiratory upstroke P2 hump artefact, i.e. during the expiratory baseline P1, and so can be used to identify hump artefacts that would otherwise have been missed. For example, in FIG. 6C the S-G filter increases and then plateaus for a short time at an inflection point before increasing again, indicating the smaller (relative to that of FIG. 6B) hump artefact in the breath waveform.

When a hump artefact is identified, either with or without the use of a first order differential, it is accounted for during the determining of the transition point(s) to ensure accurate definitions of the transition point(s). For example, after identifying a hump artefact the first order differential of the breath waveform may be normalised or re-normalised based on the maximum magnitude minima/maxima of the first order differential other than the hump artefact.

To avoid unnecessary use of computing resources, the region of the breath waveform considered during these steps (e.g. the peak detection and/or prominent minima identification) can be limited. For example, the maximum value of the breath waveform is identified and the breath waveform is divided in time into a first section and a second section. The first section is a period of the breath waveform not including the maximum value of the breath waveform, and the second section is a period of the breath waveform that includes the maximum value of the breath waveform. To save computing resources and avoid searching for a hump artefact during the inspiratory phases (P4a and P4b), the steps for identifying the hump artefact (e.g. identifying local minima, or searching for hump artefacts when at least one prominent minima is identified) are performed in the first section of the breath waveform and not in the second section of the breath waveform.

It has been found that minima corresponding to hump artefacts in capnograms typically have pCO₂ values below 2 kPa. In view of this, a predetermined threshold (herein the hump artefact threshold) may be implemented to further reduce the processing resources required when searching for a hump artefact. This may be implemented in a variety of manners, for example by discarding any minima detected where the minima is above the hump artefact threshold, or by not searching for these minima above the hump artefact threshold in the first instance. The hump artefact threshold may be used instead of the first section and second section divide described above, or in combination with that sectional divide of the breath waveform.

Even if no prominent minima are identified there may still be a hump artefact(s) present in the breath waveform. For example, this could be the case in FIG. 6C depending on the peak detection method used or the techniques used to identify prominent minima. When no prominent minima are identified, the first order differential of the waveform may be used to identify whether or not the breath waveform contains a hump artefact. The first order differential is reviewed for an inflection point region where the differential may be compared to a predetermined threshold.

Preferably, in order to reduce processing resources used, the first order differential is only reviewed in the region between the start of the first order differential and the maximum value of the first order differential.

Alternatively, rather than identifying local minima of the breath waveform and identifying prominent minima from the local minima, the hump artefact(s) can be identified using local maxima of the breath waveform (and corresponding prominent maxima).

When hump artefact(s) are present, the steps of identifying and accounting for any artefact(s) should be performed before identifying the transition point(s) for the most accurate definition of the transition point(s).

The delta transition point δ may be determined using the first order differential of the breath waveform. A preferred method comprises determining the maximum magnitude point of the first order differential (i.e. the magnitude of the highest peak or lowest trough of the first order differential) and using this to define a predetermined threshold (herein referred to as the delta threshold). For example, the delta threshold may be a value equal to 10% of the maximum magnitude point of the first order differential. This delta threshold (and other thresholds) may also be found by normalising the first order differential up to 1 with respect to the maximum magnitude point of the first order differential (after accounting for any hump artefact(s). The location of the delta transition point δ in the time dimension may then be defined as the first point in time at which the first order differential of the breath waveform is above the delta threshold (after accounting for an identified hump artefact). That is, the breath waveform value corresponding to that point in time is the delta transition point δ between the expiratory baseline P1 and the expiratory upstroke P2.

The gamma transition point γ may also be determined using the first order differential of the breath waveform. A preferred method comprises determining the maximum magnitude point of the first order differential (i.e. the magnitude of the highest peak or lowest trough of the first order differential) and using this to define a predetermined threshold (herein referred to as the gamma threshold). For example, the gamma threshold may be a value equal to the negative of 5% of the maximum magnitude point of the first order differential. The method also comprises determining the minimum value of the first order differential. The location of the gamma transition point γ in the time dimension may then be defined as the first point in time, after the minimum value of the first order differential of the waveform, at which the first order differential is higher than the gamma threshold. That is, the breath waveform value corresponding to that point in time is the gamma transition point γ between the inspiratory downstroke P4a and the inspiratory baseline P4b.

The alpha transition point α may also be determined using the first order differential of the breath waveform. A preferred method comprises determining the maximum magnitude point of the first order differential (i.e. the magnitude of the highest peak or lowest trough of the first order differential) and using this to define a predetermined threshold (herein referred to as the alpha threshold). For example, the alpha threshold may be a value equal to 15% of the maximum magnitude point of the first order differential. The method also comprises determining the maximum value of the first order differential and the maximum value of the breath waveform. In some examples the maximum magnitude point of the first order differential may also be the maximum value of the breath waveform, though this is not always the case and will depend on the specific breath waveform being analysed. The location of the alpha transition point α in the time dimension may then be defined as the first point in time after the maximum value of the first order differential at which the first order differential is less than the alpha threshold. That is, the breath waveform value corresponding to that point in time is the alpha transition point α between the expiratory upstroke P2 and the expiratory plateau P3.

In some examples of the method, the alpha threshold may be adjusted. For example, when no point of the first order differential, between the maximum value of the first order differential and the maximum value of the breath waveform, is less than the alpha threshold, the alpha threshold may be increased and the values of the first order differential (between the same boundaries) can be compared against the increased alpha threshold. This process can be performed iteratively until the alpha transition point α is defined, or until an upper limit of the number of iterations performed is reached. If this upper limit is reached the breath waveform may be rejected or the analysis of the breath waveform may continue.

To save processing resources, the location of the alpha transition point α in the time dimension may alternatively be defined as the first point in time between the maximum value of the first order differential of the breath waveform and the maximum value of the breath waveform (or the beta transition point β), at which the first order differential is less than the alpha threshold. The alpha transition point α is known to be located in the breath waveform before the beta transition point β and very likely to be before the maximum value of the breath waveform, so these limitations prevent unnecessary data analysis when determining the alpha transition point α.

An alternative method for determining the alpha transition point α comprises calculating a linear line between the delta transition point δ and the maximum value of the breath waveform (or the beta transition point), and defining the alpha transition point α based on the distance between the breath waveform and the calculated line. For example, the alpha transition point α may be defined as the point of the breath waveform, between the delta transition point δ and the maximum value of the breath waveform, that is furthest from the calculated line, when the distance between the breath waveform and the line is measured using a second linear line that is perpendicular to the calculated line.

Determining the one or more transition points may also include determining the beta transition point β between the expiratory plateau P3 and the inspiratory downstroke P4a. The beta transition point β may be used to extract additional features from the breath waveform and to help determine or verify other aspects of the waveform such as the alpha transition point α and a hump artefact.

Peak detection operations may be used to determine the beta transition point β. The peak detection operations are performed to identify local maxima of the breath waveform. Prominent maxima may then be identified from the local maxima; this is particularly useful when the breath waveform is a noisy signal. Prominent maxima may be identified by comparing each maximum identified to a threshold or by comparison amongst the set of maxima. Optionally, as it is expected that the CO₂ value of the beta transition point β will be above a predetermined threshold (herein a maxima threshold) for viable breath waveforms, CO₂ values below the maxima threshold may be ignored (e.g. by removing these values or setting them equal to 0 during the peak detection) to reduce noise from values below the maxima threshold during the peak detection.

When no prominent maxima are identified, this typically indicates the breath waveform is a bad sample and so the waveform is rejected without further unnecessary processing being performed. When only a single prominent maximum is identified, the location of the beta transition point β in the time dimension may then be defined as the time point of the single prominent maximum. When a plurality of prominent maxima are identified, the most prominent maximum is determined and this is taken as the beta transition point β. A variety of methods may be used to determine the most prominent maxima; one method uses a two-step prominence-based algorithm which compares the height of each maximum to the surrounding area and, if two or more maxima are not filtered out at that stage, compares the heights of the remaining maxima. If the most prominent maximum cannot be determined, for example in a reasonable number of iterations, then the first of these prominent maxima is taken as the beta transition point β. If other transition points are known, then these points may be used to assist determining the beta transition point β. For example, the beta transition point β is before the gamma transition point γ so any anomalous prominent maxima after the gamma transition point γ will not be considered during determining of the beta transition point β.

When the beta transition point β is known, this point may be used in the above-described method in place of the maximum value of the breath waveform. That is, the location of the alpha transition point α in the time dimension may then be defined as the first point in time, between the maximum value of the first order differential of the breath waveform and the beta transition point β, at which the first order differential is less than the alpha threshold.

The beta transition point β may also be used in the alternative method for determining the alpha transition point α described above. Instead of calculating a linear line between the delta transition point δ and the maximum value of the breath waveform, the linear line is calculated between the delta transition point δ and the beta transition point β. The alpha transition point α may then be determined based on this calculated line in the same manner described above.

Similarly, when identifying a hump artefact using the method described above, the breath waveform may be divided in time into a first section not including the beta transition point β and a second section including the beta transition point β. As previously described, the steps for identifying the hump artefact (e.g., identifying local minima, or searching for hump artefacts when at least one prominent minimum is identified) are performed in the first section of the breath waveform and not in the second section of the breath waveform.

Above we have described how a first-order differential (or a higher-order differential or a combination thereof) could be used to identify a transition point and compensate for noise and artefacts algorithmically. An alternative approach could be to analyse the point-by-point difference in the samples. For example, the method would take the point-by-point difference in the series, i.e. if the series is {t₀, t₁, t₂, . . . , tₙ}, this would yield a time series of {t₁-t₀, t₂-t₁, . . . , tₙ-tₙ₋₁} that is one shorter than the original. t₀ would be added to the beginning so the final series is {t₀, t₁-t₀, t₂-t₁, . . . , tₙ-tₙ₋₁}.

That is, the difference between each value or sample represents the change in the curve of the capnogram. This can be analysed to identify the transition points, factoring in noise and artefacts in a similar manner to that above. In summary, in addition to the approach described of having a time-based smoothing filter and first-order differential, a higher-order differential or a point-by-point difference in the time-series capnogram data (i.e. the values of the curve) could also be used to identify the shape of the curve and the transition points between phases.

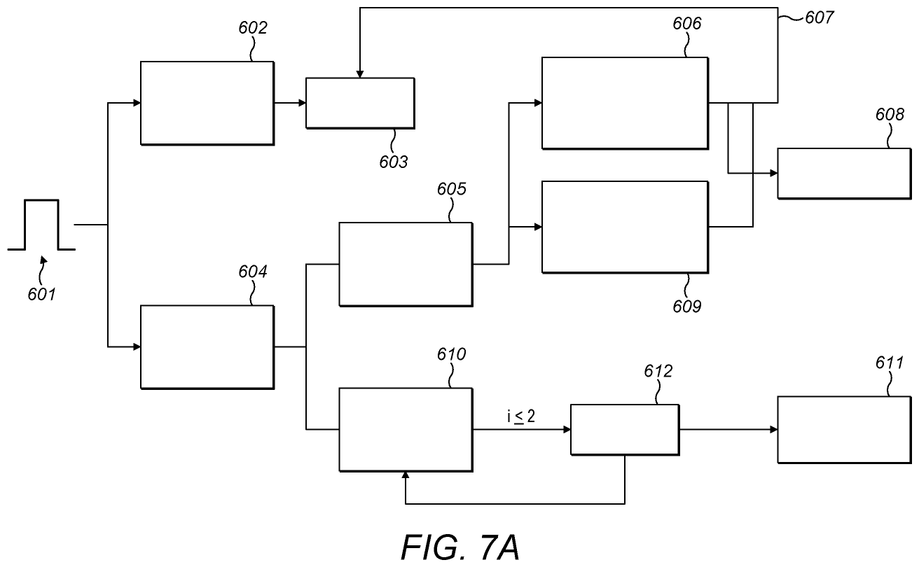

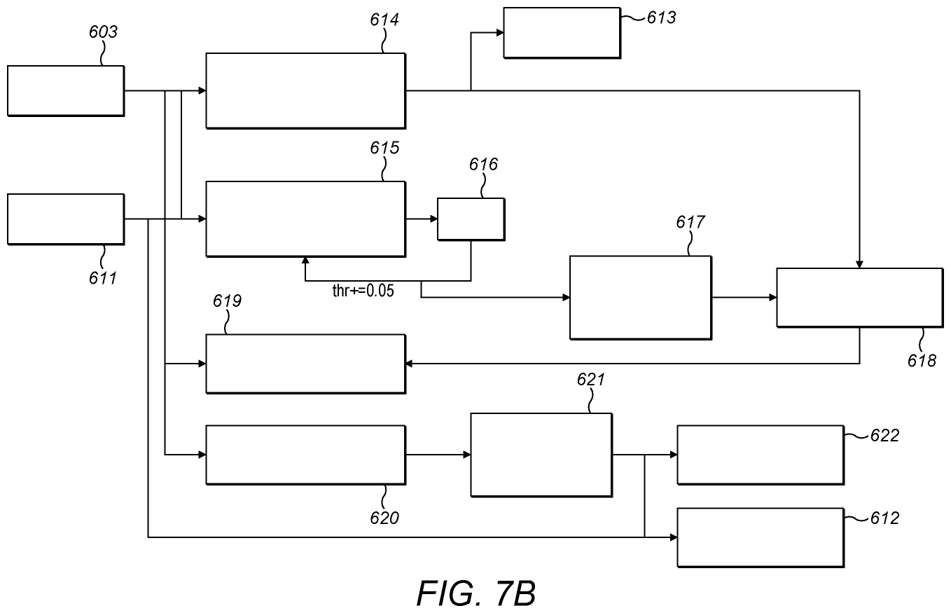

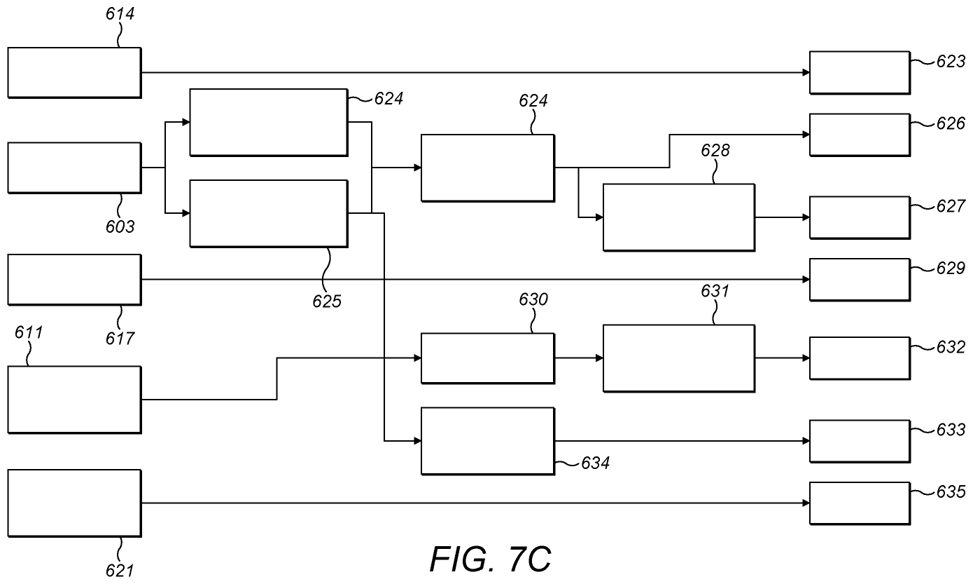

FIGS. 7A, 7B and 7C illustrate these above steps in the form of a process flow.

In a first step illustrated in FIG. 7A, the inhalation and exhalation phases are extracted by identifying the start location of the inhalation phase. The input to the process is the digitised samples of capnogram data 601. A first order Savitzky-Golay Differential is calculated using an appropriate Savitzky-Golay filter 602 and normalised 603. If this is not consistent with a good breath, it may be rejected for example by comparison with a template shape of differential or through threshold comparisons or other suitable means to identify the differential is expected.

Next, from the sampled data 601, a peak detection algorithm finds local maxima and minima 604. From a subset of the minima, if no prominent minima are detected, e.g. identified using a bidirectional local window 605, the differential is analysed 606 to identify additional humps prior to exhalation start. If these are present 608 the differential may be re-normalised 603 and the process begun again. The differential may identify no humps are present in the sampled data. If multiple minima are found in the subset, the samples are checked for a minimum in the first half of the breath, i.e. the cycle, where the minimum is less than a threshold (i.e. the hump artefact threshold). The threshold may preferably be selected as less than 2 kPA which is selected so as to identify the hump is unlikely to be on the plateau.

From the local maxima identified by the peak detection algorithm, it may be identified that the start of the inhalation is at the end of the breath. Such edge cases can be suitably processed. From a subset of the most prominent maxima 610, e.g. identified by a unidirectional forward window, the location of the start of the inhalation may be identified if only one maximum is detected 611. The waveform may be rejected if there are no maxima or if the resulting phase is too short. If there is more than one prominent maximum identified 612, the iteration described above may be performed to identify the start of the inhalation by comparing each maximum to a threshold or to each other, or alternatively taking the first maximum as the start of the inhalation phase.

In a second step, illustrated in FIG. 7B, from the normalised differential, i.e. the S-G filter differential, and the location in the waveform defined as the start of the inhalation, the method may identify the transition points and the start and end of each phase of the breath.

The location of the end of phase 1 613, may be identified as the first location in which the differential exceeds a positive threshold 614. Preferably the threshold may be 0.1 when the differential has been normalised up to 1 with respect to the maximum magnitude point of the differential (after accounting to any hump artefact(s)). The threshold is positively selected through detailed research and analysis. If the differential does not exceed the threshold, the waveform may be rejected.

From the differential, the value of the differential between the inhalation start location and the maximum point of the differential may be compared to a threshold 615. If there are too many points identified, i.e. above 20 locations, the waveform may be rejected. The threshold may be iteratively incremented 616, e.g. by 0.05, until fewer points are identified. The location 617 of the end of phase 2 can then be identified from this comparison 615. From the two identified locations 613, 617, the duration of phase 2 can be identified 618.

The end of phase 3 may be identified from the previously identified inhalation start 611. Therefore, the duration of phase 3 619 may be identified by comparing the end of phase 2 617 and the inhalation start 611.

From the differential, the first point to exceed a negative threshold between the minima of the differential and the end of the differential may be identified 620. The location of this point and the location of the minimum of the differential may thus be used to identify the end of phase 4a 621. The end of phase 4b 622 may be defined as the range of the end of phase 4a and the end of the waveform.

The duration of phase 4a 612 may be the range of the location defined as the start of the inhalation phase 611 above, i.e. the end of the phase 3, and the end of phase 4a 621.

Accordingly, the output of these two illustrated processes in FIGS. 7A and 7B, may be a normalised first order differential 603, the inhalation start location 611 and the start and end points of each phase of the waveform, i.e. the duration or ranges of the phases 613, 618, 619, 612, 622.

In a further optional example of the present disclosure, the segmentation may be further separated into angular phases. An example of such a process flow is illustrated in FIG. 7C.

The start of the delta angle 623 is defined as the end of phase 1 614. The first significant maximum 624 of the differential 603 after the local minimum 625 but before the last point in the differential attains its minimum 626 is defined as the end of the delta angle 626. The local minimum 625 is used to avoid any humps at the outset of the waveform.

The start of the alpha angle 627 is defined as the first time after the first significant maximum 624 that the differential goes below a threshold 628, preferably selected in the range from 0.2 to 0.9, and more preferably as 0.5. The end of the alpha angle 629 is defined as the end of phase 2.

The start of the beta angle is defined as the inhalation start location. The end of the beta angle 632 is identified as the first time after the inhalation start that the differential is lower than a negative threshold 631, preferably selected in the range from −0.4 to −1, and more preferably as −0.9.

The start of the gamma angle is defined as the first time after the minimum 625 of the differential that the differential exceeds a negative threshold 634, preferably selected in the range from −0.6 to −1, and more preferably as −0.9. The end of the gamma angle may be defined as the end of the phase 4a 621.

These angular features may be optionally used to identify further beneficial features, as discussed below, notably preferably based on the transition points.

In step S102, features of the breath waveform are extracted, preferably using one or more of the determined transition points.

Deriving important features (i.e. biomarkers) from a capnogram allows a machine learning model to be trained and configured to generate indicators of smoking history based on these features. Many different features of the breath waveform may be extracted using a variety of methods; a non-exhaustive list of examples is provided below. It should be noted that the benefits of each of these identified features may be separable from the overall method in which we present their context.

Extraction of biomarkers, or features, from capnography has been a significant point of focus in academia. These presented features are each particularly beneficial and may not hitherto have been described or calculated automatically. In any case, it has not been previously described that such features are extracted based on the transition points, nor how to effectively calculate them without manual intervention.

In a first set of features, the angle at a transition point may be determined by applying a linear fit to each phase on either side of the transition point and calculating the angle between them. For example, an alpha angle (the angle at the alpha transition point α) may be determined by fitting linear lines to the expiratory upstroke P2 and the expiratory plateau P3 and calculating the angle between them at the alpha transition point α where the fitted lines intersect. The linear lines may be fitted between two transition points (e.g., a linear line fitted to the expiratory upstroke P2 between the delta transition point δ and the alpha transition point α), or between the subject transition point (in this case the alpha transition point α) and another point along the adjacent phase (e.g., in this case a point along the expiratory upstroke P2 or the expiratory plateau P3). The nature of the linear fit may depend on the shape of the breath waveform. It has been found that different features are indicative of different physical characteristics. For example, in the context of the features and transition points as defined in this document, the alpha angle can be indicative of a high number of pack years.

As described above in relation to FIG. 7C, angle start and finish values (e.g., the time value and CO₂ value) are also important features that have been discovered to consistently drive the machine learning models effectively and improve their performance, and lead to further features which also improve the machine learning models. Further examples of these features are described below.

The delta angle start may be defined as the delta transition point δ (i.e., the end of the expiratory baseline P1) while the delta angle finish requires further calculation to determine. The first order differential of the breath waveform may be used to determine the delta angle finish. The location of the delta angle finish in the time dimension may then be defined as the point in time (from the start of the breath waveform or, when identified, after a hump artefact) corresponding to the first significant maximum of the first order differential of the breath waveform. Alternatively, the delta angle finish may be defined as the first significant maximum (i.e. relative to a predetermined threshold) of the differential after the local minimum before the location of the minima of the differential.

The location of the alpha angle start in the time dimension may be defined as the first point in time, after the first significant maximum of the first order differential (i.e., the first point after the delta angle finish), at which the first order differential of the breath waveform is below a predetermined threshold (herein referred to as the alpha start threshold). For example, the alpha start threshold may be a value from 20% to 90% of the maximum magnitude point of the first order differential, and more preferably is 50% of the maximum magnitude point of the first order differential. The alpha angle finish may be defined as the alpha transition point α (i.e., the end of the expiratory upstroke P2).

The beta angle start may be defined as the beta transition point β (i.e., the start of the inspiratory downstroke P4a). The location of the beta angle finish in the time dimension may be defined as the first point in time after the beta angle start that the first order differential of the breath waveform is below a predetermined threshold (herein referred to as the beta finish threshold). For example, the beta finish threshold may be a value from 40% to 100% of the negative of the maximum magnitude point of the first order differential of the breath waveform, and more preferably is 90% of the negative of the maximum magnitude point of the first order differential of the breath waveform.

The location of the gamma angle start in the time dimension may be defined as the first point in time, after the minimum value of the first order differential of the breath waveform, at which the first order differential is higher than a predetermined threshold (herein referred to as the gamma start threshold). For example, the gamma start threshold may be a value from 60% of the negative of the maximum magnitude point of the first order differential of the breath waveform, and more preferably is 90% of the negative of the maximum magnitude point of the first order differential of the breath waveform. The gamma angle finish may be defined as the gamma transition point γ (i.e., the end of the inspiratory downstroke P4a).

In step S103, an indicator of smoking history is generated based on the one or more extracted features, preferably by applying a trained machine learning model to the extracted features.

Examples of applicable machine learning algorithms include logistic regression, a gradient boosted decision trees, a support-vector machine, AdaBoost, and a random forest, though it will be appreciated other algorithms are also suitable. Some machine learning models, such as the logistic regression model, allow for the influence of each extracted feature on the prediction to be examined. Some models, such as the gradient boosted decision tree model, maintain a high level of interpretation while being resilient to overfitting, helping to ensure the model is generalisable to new data. It will be appreciated that the invention can be implemented using a variety of machine learning models other than those listed in the description.

The trained machine learning model may be configured to provide additional information together with the indicator of smoking history. For example, the model may provide a level of certainty associated with the indicator and/or highlight which extracted features contributed to the generated indicator and to what extent the (different) feature(s) contributed.

As part of step S103, the trained machine learning model may be applied to features extracted from a single breath waveform or from a plurality of breath waveforms. The plurality of breath waveforms may be from a single capnogram (i.e. a series of breath waveforms recorded by a user in a single period of breathing) or from a plurality of capnograms taken at different times. When features are extracted from a plurality of capnograms, these capnograms may be obtained (i.e. recorded by the user) over an extended period of time. For example, capnograms may be repeatedly recorded by a user several times in a day for a period of a plurality days, weeks or months. Extracting features from multiple breath waveforms obtained over an extended time period (i.e. multiple capnograms as opposed to from a breath waveform(s) from a single capnogram) allows the variability of the extracted features to be determined and provide a more accurate indicator of the user's smoking history. For example, the time of day that a capnogram is produced from a user can have an impact, as hormonal levels, such as cortisol levels, change throughout the day.

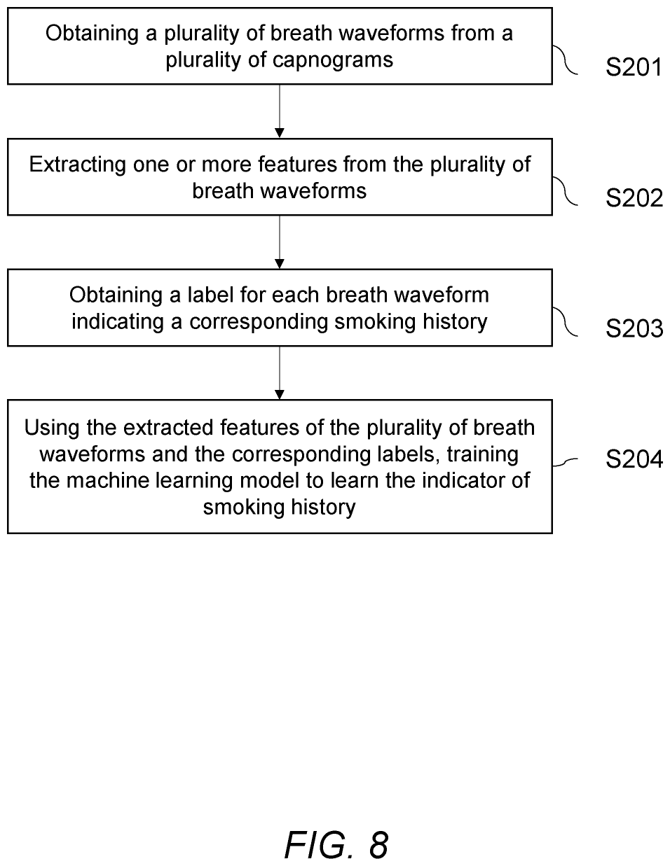

FIG. 8 shows a method for training a machine learning model to learn an indicator of smoking history.

In step S201, one or more breath waveforms are obtained from a plurality of capnograms. Step S201 mirrors step S101 by splitting a capnogram into sections consisting of a single, full breath waveform. Using a larger quantity of data with a wider variety of properties to train a machine learning model typically leads to a better trained model capable of generating more accurate indicators. Preferably, the greater quantity of data is provided by obtaining breath waveforms from a greater number of different capnograms (i.e. most preferably different capnograms produced at different times by different respiratory tracts), however a plurality of breath waveforms may also be obtained from a single capnogram of the plurality of capnograms.

In step S202, one or more features are extracted from the plurality of breath waveforms. Preferably, one or more transition points of the waveform are determined using the same techniques described above with reference to FIG. 5. This also includes, for example, determining the first order differential of a breath waveform, and identifying and accounting for hump artefact(s). Step S202 corresponds to step S102 described above and may be performed using the same techniques.

When repeating step S202 for each of the different breath waveforms of the plurality of capnograms, it is not required for the same transition point(s) to be determined for each breath waveform and/or for the same features to be extracted from every waveform. However, it is preferred that the same transition point(s) are determined and the same features extracted for a plurality of breath waveforms (and more preferably, for most of the breath waveforms) in order to more effectively train the machine learning model.

In step S203, a label is obtained for each breath waveform indicating a corresponding smoking history.

It will be apparent that step S203 may be performed before or after step S201 and/or step S202.

In step S204, the labels and the extracted features of the plurality of breath waveforms are used to train a machine learning model to learn the indicator of smoking history, preferably by creating a predictive function. The resulting predictive function is a trained machine learning model suitable for use in step S103.

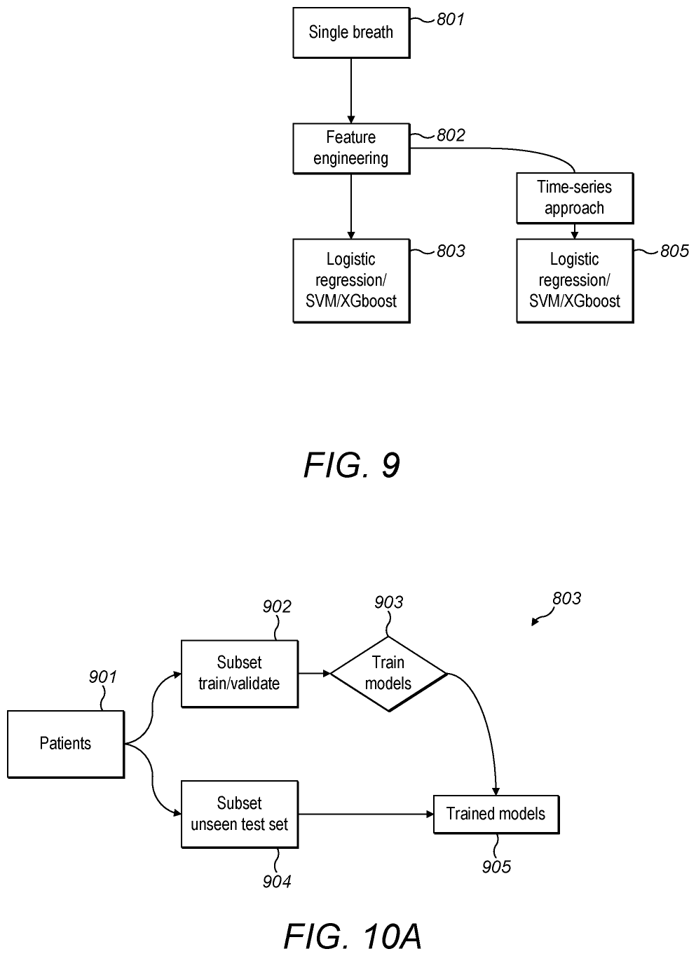

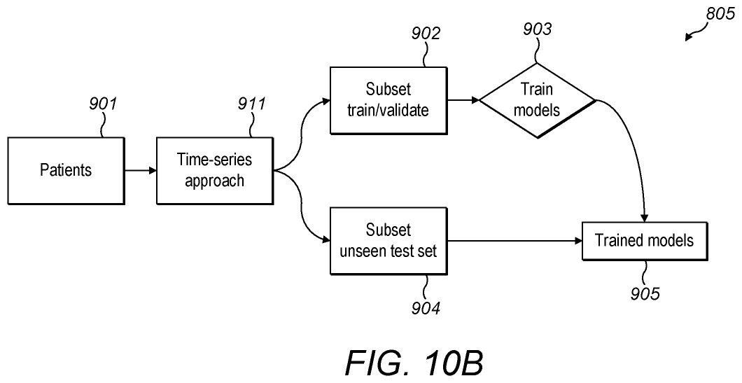

In preferred examples, multiple machine learning approaches may be combined as illustrated schematically in FIG. 9. FIGS. 10A and 10B illustrate the three components of FIG. 9.

Based on a representation of a single breath waveform, including for example an average waveform or templated waveform, 801, feature engineering 802 takes place as set out below. Multiple machine learning models are then used in two example components 803, 805 of a system.

Three different ML models were selected in this example, namely logistic regression (LR), gradient boosted decision trees (XGBoost), and the support vector machine (SVM). LR and XGBoost were chosen as simple but effective and interpretable algorithms, while the SVM, and more specifically the kernel SVM, were included to classify on a higher-dimensional, non-linear feature space. Deep learning methods could also be used however in these examples may not be preferred so as to maintain interpretability and to avoid escalation of computational complexity. It will be understood that deep learning such as an ANN or a BiLS™ may also be applicable for use with the present methods and in particular with the present feature engineering.

FIG. 10A illustrates a schematic for the implementation of generating an indicator of smoking history from a single representation of a breath record. The patients 901 are subset into training and validation data sets 902 upon which models are trained 903. Optionally, the capnograms are obtained and grouped according to a patient stratified k-fold with each patient's recordings existing within only a single fold of this splitting. A further subset 904 can optionally be used as an unseen test set input to the trained models 905 learned to classify recordings based on an indicator of smoking history.

FIG. 10B illustrates a further extension in which the task may be extended using a time-series approach to examine changes in features over time. This longitudinal information can then be used to generate an indicator of smoking history.

Longitudinal information may include multiple breath records from the same capture sessions, or multiple capnograms captured over time. A time-series approach is introduced, calculating the variability over an n day window where the variability is defined as the standard deviation between all the features extracted. As shown in FIG. 10B data is captured from the patients over a period of time. As indicated above, in examples, features extracted from each waveform at each time may be compared, for example to identify mean, median or standard deviation of the features or alternatively a statistical distribution over time of the features. These may be used to train the models further based on the variability of the features.

As shown in FIG. 10B, the patients 901 undergo a time-series approach 911 before being subset into training and validation data sets and unseen test data sets. The trained models 905, once trained 903, then classify patient data based on the learned indicator of smoking history.

Above, an algorithmic approach to identifying the transition points of the capnogram S102 has been described. In an alternative implementation, the transition points may be identified S102 using a machine learning approach.

To train a model, a plurality of breath waveforms may be obtained. As above, this may be in the form of a set of discrete samples. A set of labels may be applied to one or more samples of each breath waveform. The labels may represent the features that the machine learning model is intended to learn. For example, the label may correspond to a phase of the particular breath waveform. Alternatively, labels may correspond to a positive or negative indication of whether a sample corresponds to a transition point. Alternatively, labels may correspond to a real number that indicates how far along the breath a particular transition point occurs. In a preferred example, there may be five output classes, the labels each identifying to which of the five classes the sample belongs.

The labels and set of training breath waveforms may each be provided to a classifier or other suitable machine learning model, such as a convolutional neural network, which may be configured to learn to predict the transition points for an unseen set of samples. In a particular example, a logistic regression model can be trained in a supervised manner on the defined labels and samples so as to learn to classify each individual sample into the set of defined output classes. The input may be a whole breath standardised to a fixed length. Similarly, there may be a plurality of logistic regression models, one for each sample.

In an implementation phase, a set of unseen samples of a breath may be provided to a model (or a plurality of models in the example above where there is a trained model for each sample) trained on the above labels and training breath waveforms. The unseen samples are classified into the defined output classes. Based on the samples that neighbour the borders of each class, the transition points between phases of the unseen waveform can be identified. This information can then be used to extract features of the waveform, as identified elsewhere in this disclosure.

Another way to extract features from one or more capnograms produced from a user is to use a plurality of breath waveforms to generate a single, smooth, average breath waveform from which features can then be extracted. The extraction of features from this average breath waveform can be carried out instead of or in addition to the extraction of features from individual breath waveforms.

First, a plurality of breath waveforms from one or more capnograms produced from the user are obtained. While these breath waveforms could be provided as inputs for the method, in many instances the method will involve processing the one or more capnograms to obtain the plurality of breath waveforms. This will typically involve separating the one or more incoming capnograms into a plurality of capnogram sections. The capnogram sections will preferably each represent a single breath waveform corresponding to a single respiratory cycle and the method will therefore involve determining the delta and gamma transition points for each capnogram section in any of the manners described above, with the portion of the capnogram section between the delta transition point and the gamma transition point then extracted to generate a breath waveform. Alternatively, the cut-off points could be varied to capture more of the baseline, which can allow for more information relating to a user's respiratory cycle to be captured. It is also possible for the breath waveform to capture just a portion of a capnogram produced from the user, such that the breath waveform captures a portion of a respiratory cycle rather than a whole respiratory cycle.

The plurality of breath waveforms will typically be of different durations, reflecting the fact that a user's respiratory cycle will vary in length from one breath to another. Likewise, the CO₂ respired will vary between respiratory cycles, leading to different breath waveforms having different amplitudes. Since these differences between the breath waveforms of a single user will often not be indicative of smoking history, it is preferable for the average breath waveform not to reflect the differences in duration of the plurality of breath waveforms and, optionally, the differences in amplitude of the plurality of breath waveforms. Instead, in embodiments of the present invention the average breath waveform is used to represent the average shape of a breath waveform of the user.

To this end, the duration and, optionally, amplitude of the plurality of breath waveforms are normalised to generate a plurality of normalised breath waveforms. By duration of a breath waveform we mean the time from the start of a respiratory cycle and the end of a respiratory cycle. As will be discussed below, the start and end points can be determined in different ways, but in all cases these points are selected consistently across all breath waveforms. When normalising, the duration of each of the plurality of breath waveforms is scaled such that the duration is the same for all breath waveforms.

As with the normalisation of the duration of the plurality of breath waveforms, the amplitude of the plurality of breath waveforms may be optionally scaled such that the amplitude is the same for all breath waveforms. This amplitude can be determined in different ways, and could be as simple as the difference in CO₂ readings between the highest and lowest points of a breath waveform. In the ideal case, the highest CO₂ reading will be the end-tidal CO₂ value, and this will indeed be the case for many breath waveforms. However, the maximum amplitude will often be found at a different point of a breath waveform, either due to measurement errors or to underlying conditions of the corresponding respiratory cycle. This can lead to inconsistency in how the breath waveforms are normalised, so it is preferable to use the end-tidal CO₂ value to normalise the breath waveforms. This involves extracting an end-tidal CO₂ value from each breath waveform in any of the manners described above (e.g., determining the beta transition point) and then adjusting the amplitude of each of the plurality of breath waveforms such that each of the plurality of breath waveforms has the same end-tidal CO2 value.

Once the breath waveforms have been normalised, an average breath waveform is generated from the plurality of normalised breath waveforms, preferably using a generalised additive model (GAM). While other averaging methods, such as taking the geometric mean or the median of the plurality of breath waveforms, are possible, using a GAM allows a more physiologically accurate waveform to be generated which is not overfitted to the data from the plurality of breath waveforms. It is also more resilient to outliers in this data.

Another advantage, which will be discussed in more detail with respect to the specific example set out below, is that typically the plurality of breath waveforms each comprise a series of data points. Simply taking the geometric mean or median of the data points from the plurality of breath waveforms would therefore lead to an average breath waveform which itself comprised a discontinuous set of data points. In contrast, the use of a GAM leads to a functional representation of a breath which is continuous, thereby allowing for the average breath waveform to be used at any resolution desired by an operator.

Nevertheless, in some embodiments of the invention other methods are used to generate the average waveform.

Features can then be extracted from the average breath waveform in a similar manner to how features are extracted from a single breath waveform, and a trained machine learning model can be applied to these features to generate an indicator of smoking history. A machine learning model can be employed which makes use of features extracted from individual breath waveforms of the user when generating an indicator of smoking history in addition to the features extracted from the average breath waveform. Alternatively, a machine learning model can be employed which makes use of features extracted from either individual breath waveforms of the user or the average breath waveform alone when generating an indicator of smoking history.

The usefulness of the average breath waveform generated, whether this has been generated using a GAM or using some other method, can be improved further still by first identifying whether any of the plurality of normalised breath waveforms are anomalous and then excluding said anomalous normalised breath waveforms before generating the average breath waveform.

One advantageous way to identify anomalous normalised breath waveforms is to interpolate the normalised breath waveforms to all have the same number of data points, and to directly compare the data points of one normalised breath waveform with the data points from the other normalised breath waveforms. Any normalised breath waveforms having one or more anomalous data points are then identified as being anomalous and excluded.

This approach has been found to be less computationally intensive than other approaches to excluding anomalous breaths. For example, interpolating the normalised breath waveforms and comparing data points to identify anomalous breaths has been found to be less computationally intensive than standard anomaly detection algorithms.

A specific example of this method will now be described which has been found to be particularly advantageous.

This example has been described in the case of breath waveforms obtained from a single capnogram, but it could equally be applied in the case of breath waveforms obtained from multiple capnograms. Likewise, the skilled person will understand that other modifications of this specific example are possible. For example, the example described below could be modified to use breath waveforms capturing different portions of a respiratory cycle than the waveform between the delta transition point and the gamma transition point, such as those which capture more of the baseline or those which capture a predefined portion of a respiratory cycle rather than a whole respiratory cycle.

Firstly, a capnogram is obtained and then separated into M constituent capnogram segments, whereafter only the waveform between each of the delta transition point and the gamma transition point is extracted for each capnogram segment. These transition points may be identified in any of the manners discussed above, for example such that the waveform is extracted between the delta angle start and the gamma angle end for each capnogram segment. The use of the delta and gamma transition points is especially advantageous as this results in an average breath waveform that is more similar to a single breath waveform extracted from a capnogram. As such, features that can be extracted from a single breath waveform may also be extracted from an average breath waveform. Furthermore, in embodiments in which a machine learning model is to be employed which makes use of features extracted from individual breath waveforms of the user when generating an indicator of smoking history in addition to the features extracted from the average breath waveform, these transition points may have already been identified for one or more of the breath waveforms.

The breath waveforms are numbered from 1 to M, with breath waveform m being defined as the pair (tₘ, yₘ), where tₘ is the vector of timestamps for each CO₂ partial pressure value in the vector yₘ and having length Nₘ. All breaths are then individually normalised to the same duration and optionally height (which is to be understood as meaning the same as amplitude), whilst maintaining its unique shape. The optional height normalisation may be achieved by scaling the end-tidal CO₂ (which may be calculated in any of the manners described above) value to 5 KPa, so that

$${\tilde{y}}_{m} = \frac{5}{{y}_{m , end - tidal}} {y}_{m} ∀ m ∈ \{ 1 , … , M \} .$$

The time series points are shifted such that they begin at 0, and are then scaled linearly such that the final point is at 3 seconds,

$${\tilde{t}}_{m} = \frac{3}{{t}_{m} , {N}_{m}} ( {t}_{m} - {t}_{m , 1} ) ∀ m ∈ \{ 1 , … , M \} .$$

The skilled person will of course understand that other values of the scaled end-tidal CO₂ value could be chosen, as could other values of the scaled duration. As has been discussed above, while optional, it is advantageous in some instances to exclude anomalous normalised breath waveforms. In the present example, this is done by linearly interpolating all (normalised) breath waveforms to the same length (which is to say to all comprise the same number of data points), to give (t̅ₘ, y̅ₘ)∀m∈{1, . . . , M} and |t̅ₘ|=|y̅m|=N:=400 ∀m∈{1, . . . , M}. The skilled person will understand that other values of N could be chosen. This allows the nth data point of each (normalised) breath waveform to be directly compared with the nth data point of every other (normalised) breath waveform. Breath waveforms for which one or more of these data points are found to be significant outliers can then be excluded as being anomalous.

In the present example, any breath waveforms m∈{1, . . . , M} having at least one data point n∈{1, . . . , N} for which y̅_{m,n} is further than 3 standard deviations from the median (i.e. the median of the nth data points of every breath waveform) are excluded, leaving a subset of the original set of normalised breath waveforms, namely M̅. The skilled person will understand that a different threshold for abnormal breath waveforms could be chosen. For example, a different number of standard deviations could be chosen as the threshold, or alternatively a different measure could be chosen, such as a percentage difference from the median.

Using this set of (t̅ₘ, y̅ₘ)∀m∈M̅, a generalised additive model (GAM) is then used to generate a single, smooth, average breath for the capnogram. In this specific example, the GAM is defined as E[y|t]=ƒ(t), which aims to minimise

$$\frac{1}{N \vert \overline{M} \vert} {\sum }_{n = 1}^{N} {\sum }_{m ∈ \overline{M}} {( {\overline{y}}_{m , n} - f ( {\overline{t}}_{m , n} ) )}^{2} + λ {\int }_{t = 0}^{3} {{f}^{″} ( t )}^{2} dt$$

The final term provides a cost for excessive curvature, and the parameter λ therefore controls the level of penalisation due to excessive curvature, thereby limiting the degree of overfitting. The skilled person will understand that this term is optional and may be omitted by setting λ=0. Although other processes such as Gaussian Process Regression or kernel ridge regression could be used, these are more computationally intensive or produce less smooth curves than a GAM and take a long time to fit for high resolution average breath waveforms.

This function to be minimised can be rewritten by concatenating all the (t̅ₘ, y̅ₘ)∀m∈M into a single set (t̅, y̅) where each of the vectors are of length |t̅|=|y̅|=N|M̅|, and minimising

$$\frac{1}{N \vert \overline{M} \vert} {\sum }_{i = 1}^{N \vert \overline{M} \vert} {( {\overline{y}}_{i} - f ( {\overline{t}}_{i} ) )}^{2} + λ {\int }_{t = 0}^{3} {{f}^{″} ( t )}^{2} dt$$

This model is fitted by learning the transformation function ƒ, using the set of breaths (t̅, y̅).

In order to fit the model, a class of functions from which ƒ should belong is first chosen. In the present case, this is the set of cubic splines. These are the set of piecewise cubic polynomial functions that interpolate between specified control points (knots) while matching the zeroth, first and second derivatives at these knots, forming a single, smooth, fit with continuity down to the second derivative (as shown in figure). The cubic spline is built up using a linear combination of the set of basis-splines, ϕⱼ(t), to give

$$f ( t ) = {\sum }_{j = 1}^{J} {β}_{j} {ϕ}_{j} ( t )$$

where the coefficients, βⱼ, are fitted by the minimisation and ϕⱼ(t). The specific form of ϕⱼ(t) are known in the art, and are distributed uniformly in time.

The number J is specified, where the first and last basis functions are positioned at the first and last times, such that they can all be scaled horizontally to fit within the 0 to 3 second window. (As noted above, the breath waveforms could be normalised to different durations, in which case the basis functions would also be scaled to a different duration.) The coefficients are fitted with Poisson Iteratively Reweighted Least Squares (PIRLS) iterations. It has been found that J=100 allows any kind of breath (i.e. healthy or non-healthy) of any length to be fitted well. The skilled person of course understands, however, that different fitting and optimisation procedures could be used.

The final numerical average waveform output is yₙ=ƒ(tₙ) where tₙ is a series of time points in the range [(t̅), (t̅)], which in this example means

$${t}_{n} = 3 \frac{n - 1}{N - 1} ∀ n ∈ \{ 1 , … , N = 400 \} .$$

(As noted above, a different value of N could be chosen.) Using this average breath waveform, features may be calculated using the methods described above.

The advantage of using splines is that this approach is more computationally efficient than other approaches, for example fitting a high-order polynomial. Likewise, cubic basis splines have been found to represent the best trade-off between ensuring continuity of the GAM up to the second order derivative and computational complexity. Lower order splines will be discontinuous at the second (and possibly also first) derivative, and this discontinuity in curvature reduces the effectiveness of feature extraction since some features are based on the curvature parameters of the average breath waveform. Higher order splines do not have this issue, but they increase the computational complexity in fitting the GAM and can also lead to oscillations in the average waveform which do not reflect the underlying data.

One alternative is to use Bezier curves instead of splines. However, using Bezier curves without the use of knots is significantly more computationally intensive, while conversely using knots with Bezier curves will lead to discontinuities in the first derivative. Both of these are significant disadvantages as compared with the present method.

As has been noted, other modifications to the example given above are possible. One modification relates to how the breaths are normalised.

While the duration and height of the breath waveforms may be normalised to arbitrary values, in some preferable embodiments the breaths are normalised to the average end-tidal CO₂ value of the series of breath waveforms, with the average end-tidal CO₂ value defined as follows:

$${y}_{end - tidal} := \frac{1}{M} {\sum }_{m = 1}^{M} {y}_{m , end - tidal}$$

This leads to the following normalised height for each breath waveform:

$${\tilde{y}}_{m} = \frac{{y}_{end - tidal}}{{y}_{m , end - tidal}} {y}_{m}$$

Similarly, the duration of each breath waveform may be normalised to the average duration of the series of breath waveforms. This is particularly advantageous when the normalisation step is combined with extraction of the breath waveforms from their respective capnogram segments, rather than receiving the breath waveforms after this extraction has occurred.

In this approach, the timestamp of the delta angle, t_{m,d}, and the timestamp of the gamma angle t_{m,g} are identified for each breath waveform, with the difference between these Δtₘ=t_{m,g}−t_{m,d} defining the length of the portion of the breath waveform to be used when generating the average breath waveform. The average of these values is then taken to determine an average delta time, an average gamma time, and an average difference:

$${t}_{d} := \frac{1}{M} {\sum }_{m = 1}^{M} {t}_{m , d}$$

$${t}_{g} := \frac{1}{M} {\sum }_{m = 1}^{M} {t}_{m , g}$$

$$Δ {t}_{μ} := \frac{1}{M} {\sum }_{m = 1}^{M} Δ {t}_{m}$$

(Although the arithmetic mean has been used as the average, other measures of the mean could be used, as could the median.)

The timestamps of each of the breath waveforms is then translated as follows:

$${t}_{m} := \frac{Δ {t}_{μ}}{Δ {t}_{m}} ( {t}_{m} - {t}_{m , d} ) + {t}_{d} .$$

Here, the first term translates and rescales the breath waveform so that the delta transition point is at 0 seconds and the time difference between the delta and gamma transition points equal to the average Δt_{μ}. The second term then translates the breath back so that the delta transition point for this breath is at the timestamp of the average delta transition point. (As noted above, the breath waveforms can be normalised to any arbitrary value, such that Δt_{μ}=3 for a length of 3 s).

At this point, the delta transition points and gamma transition points are synced between the breath waveforms. However, each breath waveform will typically comprise a portion of the baseline, and the amount of this baseline will vary between the breath waveforms. This means that the start and end times of the breath waveforms will differ. In order to address this, the breaths are cut to a predefined length. This can be arbitrary, but to extract breaths between the delta transition point and the gamma transition point this preferably involves the following steps.

First, the average duration of the breath from the gamma transition point to the breath end is identified. For example, where the arithmetic mean is used to define the average difference:

$$Δ {t}_{g} := \frac{1}{M} {\sum }_{m = 1}^{M} {t}_{m , {N}_{m}} - {t}_{m , g}$$

where t_{m,N}_{ₘ} is the timestamp of the end point of breath m.

Each breath waveform is then cropped at its closest timestamp to t_{g}+Δt_{g} to define (\tilde{t}ₘ, \tilde{y}ₘ) where, as before, |\tilde{t}ₘ|=|\tilde{y}ₘ|=Ñₘ. (Alternatively, each breath waveform could be cropped to the average breath duration such that (when using the arithmetic mean)

$${\tilde{t}}_{m , {N}_{m}} = \frac{1}{M} {\sum }_{m = 1}^{M} {t}_{{N}_{m}} ∀ m ∈ \{ 1 , ... , M \} . )$$

Any breaths that are smaller than the chosen cropping length are extended by either copying their final value at regular intervals until the cropping length, or by a linear fit until the chosen cropping length.

Another modification to the example given above relates how to the optional step of excluding anomalous breaths is performed. In some embodiments the breath waveforms are interpolated to the same length without excluding anomalous breaths. In other embodiments no interpolation is performed and nor are anomalous breaths excluded. In those embodiments where the breath waveforms are interpolated and anomalous breaths excluded, it can be advantageous to revert the non-excluded breaths back to their original non-interpolated form after the exclusion of anomalous breaths.

The generation of the average waveform may also vary from the approach described above. While it is advantageous to use a GAM for this, this need not use splines to learn the transformation function ƒ. For example, linear and factor terms could also be used. While it has been found that linear terms on their own do not typically produce a good fit, factor terms lead to a better fit, although this fit is typically more jagged than when using splines. Any combination of spline terms, factor terms, or linear terms could therefore be used.

For example, when including these additional terms the function fitted by the GAM is

$$f ( t ) = {\sum }_{j = 1}^{J} {β}_{j} {ϕ}_{j} ( t ) + {\sum }_{j = 1}^{{J}_{α}} {α}_{j} {φ}_{j} ( t ) + {\sum }_{j = 1}^{{J}_{γ}} {γ}_{j} {ψ}_{j} ( t )$$

where αⱼ for j∈{1, . . . , J_{α}} and γⱼ for j∈{1, . . . , J_{γ}} are coefficients and J, J_{α}, J_{β} are arbitrary values selected when fitting the transformation function ƒ. The basis functions of the factor terms, φⱼ, and the basis functions for the linear terms, ψⱼ, will of course be known to the skilled reader. Typically |βⱼ|>>|αₖ|∀j∈{1, . . . , J}, k∈{1, . . . , J_{α}} and |βⱼ|>>|γₖ|∀j∈{1, . . . , J_{γ}}, k∈{1, . . . , J_{γ}}, i.e. the weights assigned to spline terms are much greater than for the linear or factor terms, due to the better fitting provided by the spline terms.

It is also possible to generate the average waveform without using a GAM.

One such example generates an average waveform using the average of the CO2 values at each time point. For example, when using an arithmetic mean:

$${y}_{i}^{*} = \frac{1}{\vert \overline{M} \vert} {\sum }_{m = 1}^{\overline{M}} {\overline{y}}_{m , i} ∀ i ∈ \{ 1 , ... , N \}$$

and, furthermore, a standard deviation can be calculated at each time point:

$${σ}_{i}^{*} = \sqrt{\frac{1}{\vert \overline{M} \vert} {\sum }_{m = 1}^{\overline{M}} {( {\overline{y}}_{m , i} - \frac{1}{\vert \overline{M} \vert} {\sum }_{m = 1}^{\overline{M}} {\overline{y}}_{m , i} )}^{2}} ∀ i ∈ \{ 1 , ... , N \}$$

As mentioned above, although they are more computationally intensive or produce less smooth curves than a GAM, two other possible approaches are Gaussian process regression and kernel ridge regression.

Similar to the GAM approach, these approaches start with a pre-processing of the received breaths to arrive at breath waveforms (t̅, y̅). Kernel ridge regression then aims to find the weight vector α that minimises

$${\sum }_{i = 1}^{\vert \overline{t} \vert} L ( {y}_{i} , {( K α )}_{i} ) + {λα}^{T} K α$$

where L is a loss function and K is the kernel matrix Kᵢⱼ=k(t̅ᵢ, t̅ⱼ) built using the kernel function, k(x, y), where for example

$$k ( x , y ; σ ) = {e}^{- \frac{{‖ x - y ‖}_{2}^{2}}{2 {σ}^{2}}}$$

is the RBF kernel. Similar to the GAM implementation, λ acts as a smoothing parameter that may be set for when an optimisation algorithm tunes α.

Then, the average waveform is generated using

$${y}_{n} = {\sum }_{i = 1}^{\vert \overline{t} \vert} k ( {\overline{t}}_{i} , {t}_{n} ) {α}_{i}$$

where tₙ is a series of time points in the range [(t̅), (t̅)].

After preprocessing of the received breaths to arrive at breath waveforms (t̅, y̅), Gaussian process regression starts by defining the new time points of the average waveform as a series of time points in the range [(t̅), (t̅)], that are referred to as t* in the following.

The Gaussian Process Regressor model will be known to the skilled person, and in this implementation makes use of a kernel function specified similarly to the kernel ridge regression model that will form kernel matrices whose elements are defined as

$${K}_{ij} = k ( {\overline{t}}_{i} , {\overline{t}}_{j} )$$

$${K}_{ij}^{*} = k ( {t}_{j}^{*} , {\overline{t}}_{i} )$$

$${K}_{ij}^{* *} = k ( {t}_{i}^{*} , {t}_{j}^{*} )$$

Using a hyperparameter σ_{ε}, the average waveform is predicted as

$$y *= K * {( K + {σ}_{ε}^{2} I )}^{- 1} \overline{y}$$

and the standard deviations given by the vector

$$σ *= \sqrt{diag ( K ** - K * {( K + {σ}_{ε}^{2} I )}^{- 1} K * )}$$

(where the square root is element-wise) and I is the identity matrix.

An extra optional but preferable step is to automatically select hyperparameters which are those of the kernel function and σ_{ε}. This is done by minimising the negative log marginal likelihood using a standard optimisation algorithm:

$$p ( y \vert X ) = \frac{1}{2} {{\overline{y}}^{T} ( K + {σ}_{ε}^{2} I )}^{- 1} \overline{y} + \frac{1}{2} \vert K + {σ}_{ε}^{2} I \vert + \frac{n}{2} 2 π$$

The hyperparameters found from this minimisation can then be inserted into the calculations above.

A variation of this Gaussian process regression framework is described in is known as sparse Gaussian process regression and, although this variation sacrifices a degree of accuracy, is more computationally efficient.

After obtaining (t̅, y̅), ‘inducing inputs’ are then specified. These inducing inputs are an additional series of t values that are smaller in size than t̅. We name these t′. A set of kernel matrices are then defined using the elements of the inducting inputs:

$${K}_{ij}^{′} = k ( {\overline{t}}_{i} , {t}_{j}^{′} )$$

$${K}_{ij}^{′′} = k ( {t}_{i}^{′} , {t}_{j}^{′} )$$

$${K}_{ij}^{′ *} = k ( {t}_{i}^{′} , {t}_{j}^{*} )$$

Further distribution parameters are also defined as follows:

$$\sum = {( {K}^{″} + \frac{1}{{σ}_{ε}^{2}} {K}^{′ T} {K}^{′} )}^{- 1}$$

$$μ = \frac{1}{{σ}_{ε}^{2}} {K}^{″} \sum {K}^{′ T} \overline{y}$$

$$A = {K}^{″} \sum {K}^{″}$$

These parameters allow

$$y *= {K}^{′ * T} {K}^{″ - 1} μ$$

$$σ *= \sqrt{diag ( K ** - {K}^{′ * T} {K}^{″ - 1} {K}^{′ *} + {K}^{′ * T} {K}^{″ - 1} {AK}^{″ - 1} {K}^{′ *} )}$$

to be computed.

(Note that the inducing inputs t′ can be chosen manually or alternatively selected automatically using an optimisation algorithm that minimises the following expression with respect to t′:

$$\frac{1}{2 {σ}_{ε}^{2}} Tr [ K - Q ] - N ( \overline{y} ; 0 , {σ}_{ε}^{2} I + Q )$$

where Q=K′K″⁻¹K′^{T}, N is a normal distribution and Tr is the trace function.)

A final alternative to using a GAM is a similar process to Gaussian process regression but which models each point in time as a “Student t” distribution rather than a Gaussian distribution.

After obtaining (t̅, y̅), a kernel function is now defined as before but with a small addition

$${σ}_{ε}^{2} I$$

as follows:

$$\tilde{k} ( x , y ) = k ( x , y ) + {σ}_{ε}^{2} I$$

This gives kernel matrices defined by the elements as

$${\tilde{K}}_{ij} = \tilde{k} ( {\overline{t}}_{i} , {\overline{t}}_{j} )$$

$${\tilde{K}}_{ij}^{*} = \tilde{k} ( {t}_{i}^{*} , {\overline{t}}_{j} )$$

$${\tilde{K}}_{ij}^{* *} = \tilde{k} ( {t}_{i}^{*} , {t}_{j}^{*} )$$

This gives us the equations to calculate the average waveform and its standard deviation as:

$$y *= {\tilde{K}}^{* T} {\tilde{K}}^{- 1} \overline{y}$$

$$σ *= \sqrt{diag ( \frac{ω + {\overline{y}}^{T} {\tilde{K}}^{- 1} \underline{y}}{v + \vert \overline{t} \vert - 2} ( \tilde{K} ** - {\tilde{K}}^{* T} {\tilde{K}}^{- 1} \tilde{K} \text{*)} )}$$

where ω, v are hyperparameters. As before, the hyperparameters, including any from the kernel function can first be chosen manually or optimised by minimising the negative log marginal likelihood:

$$\frac{v + \vert \overline{t} \vert}{2} \vert \tilde{K} + ω \overline{y} {\overline{y}}^{T} \vert - \frac{v + \vert \overline{t} \vert - 1}{2} \vert \tilde{K} \vert + {Γ}_{\vert \overline{t} \vert} ( \frac{v + \vert \overline{t} \vert - 1}{2} ) - {Γ}_{\vert \overline{t} \vert} ( \frac{v + \vert \overline{t} \vert}{2} ) + \frac{\vert \overline{t} \vert}{2} \vert πω \vert$$

where

$${Γ}_{\vert \overline{t} \vert} ( x ) ≡ {π}^{\frac{\vert t \vert ( \vert t \vert - 1 )}{4}} {\prod }_{i = 1}^{\vert \overline{t} \vert} Γ ( x + \frac{1}{2} - \frac{i}{2} )$$

and Γ is the gamma function.

Any or all steps of the methodology may be implemented in a remote or cloud computing device or locally at the edge, i.e. on a device capable of retrieving the capnogram data. In examples, training is performed centrally before the implantation or test phase is performed using a trained model stored locally either on the device, e.g. with the model parameters stored in memory on SoC, or on a local computer. The trained model and its associated parameters may be stored centrally, i.e. on the cloud. Methods and processes described herein can be embodied as code (e.g., software code) and/or data. The models, methodologies and algorithms may be implemented in hardware or software as is well-known in the art of machine learning. For example, hardware acceleration using a specifically programmed Graphical Processing Unit (GPU) or a specifically designed Field Programmable Gate Array (FPGA) may provide certain efficiencies. For completeness, such code and data can be stored on one or more computer-readable media, which may include any device or medium that can store code and/or data for use by a computer system. When a computer system reads and executes the code and/or data stored on a computer-readable medium, the computer system performs the methods and processes embodied as data structures and code stored within the computer-readable storage medium. In certain embodiments, one or more of the steps of the methods and processes described herein can be performed by a processor (e.g., a processor of a computer system or data storage system).

Generally, any of the functionality described in this text or illustrated in the figures can be implemented using software, firmware (e.g., fixed logic circuitry), programmable or nonprogrammable hardware, or a combination of these implementations. The terms “component” or “function” as used herein generally represents software, firmware, hardware or a combination of these. For instance, in the case of a software implementation, the terms “component” or “function” may refer to program code that performs specified tasks when executed on a processing device or devices. The illustrated separation of components and functions into distinct units may reflect any actual or conceptual physical grouping and allocation of such software and/or hardware and tasks.

An example of a comparison of a smoking indicator generated according to embodiments of the present invention with data collected using conventional methods is described below.

The global tobacco epidemic is considered as one of the greatest threats to public health, with tobacco smoking widely recognised as the leading contributor to respiratory diseases. The dose-response relationship between cumulative smoking, in this example tobacco smoking, and the severity of airways obstruction, in this example patients with COPD, is difficult to characterise using non-specific methods such as spirometry.

This comparison evaluates the relationship of smoking history and features of small/medium-sized airway obstruction in patients with Chronic Obstructive Pulmonary Disease (COPD) using fast-response capnography data yielded using Cambridge Respiratory Innovations' (CRI's) N-Tidal device.

305 COPD GOLD (Global Initiative for Chronic Obstructive Lung Disease) 3 or 4 subjects were recruited from three studies in the UK. Tobacco smoking data was collected at baseline; N-Tidal CO₂ data was collected twice daily for up to 6 weeks. The relationship between CO₂ features from the expiratory upstroke and plateau phases were correlated to subjects' smoking histories.

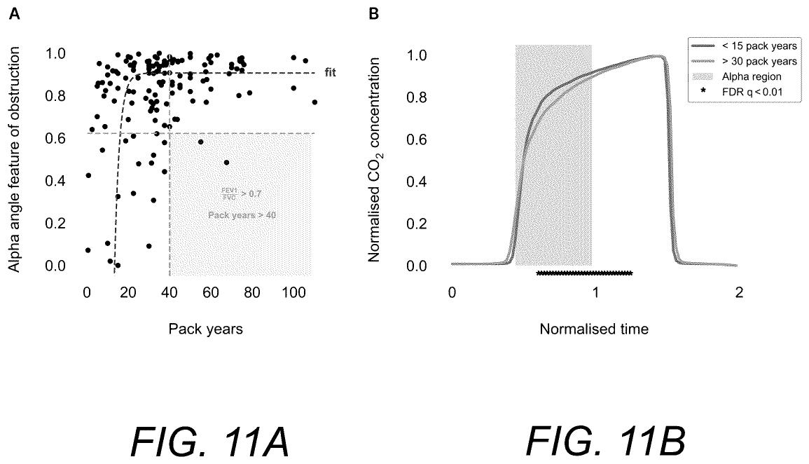

Higher smoking pack years was associated with greater curvature in the alpha-angle region, which may relate to structural airway remodelling of smaller airways. Alpha-angle features showed a significantly altered CO₂ waveform geometry beyond 40 pack years. This can be seen in FIGS. 11A and 11B which respectively illustrate a regression plot of CO₂ alpha-angle feature vs pack years and average CO₂ waveforms across subjects with <15 pack years vs >30 pack years smoking history.

N-Tidal CO₂ waveform features of airway obstruction therefore demonstrate a dose-response relationship with cumulative smoking history. N-Tidal may be able to directly probe airway remodelling as a result of smoking.

1. A method for generating an indicator of smoking history from one or more capnograms produced from a user, the method comprising:
obtaining one or more breath waveforms from the one or more capnograms produced from the user;
extracting one or more features from the one or more breath waveforms; and
generating the indicator of smoking history based on the one or more features.

2. A method according to claim 1, wherein generating the indicator of smoking history comprises using the one or more extracted features as inputs to a trained machine learning model, wherein the trained machine learning model is configured to output the indicator of smoking history;
optionally, wherein generating the indicator of smoking history further comprises using one or more pieces of demographic information as inputs to the trained machine learning model;
optionally, wherein the one or more pieces of demographic information comprise one or more of: age; sex; and ethnicity.

3. A method according to claim 2, wherein generating the indicator of smoking history further comprises using a time of day that each of the one or more capnograms were produced from the user as an input to the trained machine learning model.

4. A method according to claim 2, wherein generating the indicator of smoking history further comprises: obtaining a disease label associated with the one or more breath waveforms; and at least one of: using the disease label as an input to the trained machine learning model; and selecting the trained machine learning model from two or more trained machine learning models based on the obtained disease label.

5. A method according to claim 2, wherein the trained machine learning model comprises at least one of: logistic regression, a gradient boosting decision tree, a support-vector machine, AdaBoost, and a random forest.

6. A method according to claim 1, wherein the indicator of smoking history comprises one or more of: pack years associated with the user; and a lung age associated with the user.

7. A method according to claim 1, the method further comprising:
determining a variability of the one or more extracted features; and
using the variability of the extracted features as an input to a trained machine learning model;
optionally, wherein the one or more breath waveforms are recorded from the same user over a time period comprising two or more days.

8. A method according to claim 7, wherein the trained machine learning model is further configured to output an indication of an importance of an extracted feature that contributed to the generated indicator of smoking history.

9. A method according to claim 1, wherein the breath waveform represents a single respiratory cycle;
optionally, wherein obtaining the one or more breath waveforms comprises splitting each of the one or more capnograms into a plurality of capnogram sections, wherein each capnogram section represents a single breath waveform corresponding to the single respiratory cycle.

10. A method according to claim 1, wherein extracting one or more features from the one or more breath waveforms comprises:
normalising a duration of the one or more breath waveforms to generate a corresponding one or more normalised breath waveforms;
generating an average breath waveform from the one or more normalised breath waveforms; and
extracting the one or more features from the average breath waveform;
optionally, wherein the average breath waveform is generated from the one or more normalised breath waveforms using a generalised additive model, GAM;
optionally, the method further comprising normalising an amplitude of the one or more breath waveforms to generate the one or more normalised breath waveforms;
optionally, wherein normalising the amplitude of the one or more breath waveforms comprises:
extracting an end-tidal CO2 value from each breath waveform; and
adjusting the amplitude of each of the one or more breath waveforms such that each of the one or more breath waveforms has the same end-tidal CO2 value.

11. A method according to claim 1, wherein extracting the one or more features comprises:
determining one or more transition points of each of the one or more breath waveforms; and
extracting the one or more features using the one or more transition points;
wherein the one or more transition points comprise one or more of:
an alpha transition point between an expiratory upstroke and an expiratory plateau;
a beta transition point between the expiratory plateau and an inspiratory downstroke;
a gamma transition point between an inspiratory downstroke and an inspiratory baseline; and
a delta transition point between an expiratory baseline and an expiratory upstroke;
optionally, wherein determining the alpha transition point comprises:
identifying a maximum value of a first order differential of the breath waveform;
identifying the maximum value of the breath waveform and/or determining the beta transition point; and
defining the alpha transition point as the first point in time after the maximum value of the first order differential, between the maximum value of the first order differential and the maximum value of the breath waveform and/or the beta transition point, at which the first order differential of the breath waveform is less than an alpha threshold;
optionally, wherein determining the alpha transition point further comprises:
when no point between the maximum value of the first order differential of the breath waveform and the maximum value of the breath waveform is less than the alpha threshold, or when no point between the maximum value of the first order differential of the breath waveform and the beta transition point is less than the alpha threshold, increasing the alpha threshold;
optionally, wherein determining the alpha transition point comprises:
calculating a line between the delta transition point and the maximum value of the breath waveform, or calculating a line between the delta transition point and the beta transition point; and
defining the alpha transition point based on a distance between the breath waveform and the calculated line;
optionally, wherein determining the beta transition point comprises:
performing peak detection to identify local maxima of the breath waveform;
identifying prominent maxima from the local maxima;
when only a single prominent maximum is identified, determining this as a beta transition point; and
when a plurality of prominent maxima are identified, determining the most prominent maximum and defining this as the beta transition point;
optionally, wherein determining the delta transition point comprises:
determining the first point in time at which a first order differential of the breath waveform is above a delta threshold; and
defining the first point as the delta transition point.

12. A method according to claim 11, wherein determining one or more transition points of a breath waveform comprises determining a derivative of said breath waveform;
optionally, wherein the derivative of the breath waveform is a first order differential of the breath waveform;
optionally, wherein the method further comprises:
using the first order differential of the breath waveform to determine whether the breath waveform is an anomalous breath waveform; and,
when the breath waveform is an anomalous breath waveform, rejecting the breath waveform;
optionally, wherein determining the first order differential of the breath waveform comprises applying a time-based smoothing filter to the breath waveform;
optionally, wherein determining one or more transition points of a breath waveform comprises:
identifying a hump artefact in said breath waveform and, when there is a hump artefact, accounting for the hump artefact during the determining of the one or more transition points;
optionally, wherein identifying the hump artefact comprises:
performing peak detection to identify local minima of the breath waveform;
identifying prominent minima from the local minima;
identifying the maximum value of the breath waveform and/or determining the beta transition point;
dividing the breath waveform into a first section not including the maximum value of the breath waveform, and a second section including the maximum value of the breath waveform and/or the beta transition point;
when at least one prominent minimum is identified, searching for hump artefact(s) in the first section of the breath waveform; and/or
when no prominent minima are identified, using the first order differential of the breath waveform to search for hump artefact(s) in the first section of the breath waveform

13. A method according to claim 11, wherein determining the transition points comprises:
applying a trained machine learning model to a set of discrete samples of the breath waveform, the breath waveform representing a whole breath, wherein the machine learning model is configured to classify each sample into one of a plurality of output classes, each class representing a region of the breath waveform, and wherein the machine learning model is trained by:
obtaining a label associated with each discrete sample of a plurality of breath waveforms, each breath waveform being represented by a set of samples representing a whole breath and each label indicating which of a plurality of output classes that sample corresponds to; and
training the machine learning model on the labels and the samples to learn to classify a sample of a set of samples representing a whole breath into a class of the plurality of output classes.

14. A method according to claim 11, wherein extracting features of a breath waveform using the one or more transition points comprises determining an angle of each of the one or more transition points;
optionally, wherein determining the angle of each of the one or more transition points comprises, for each of the one or more transition points:
fitting a first linear function and a second linear function to the adjacent phases on either side of the transition point and measuring the angle between the first and second linear functions; and/or
fitting a third linear function to the expiratory upstroke or the inspiratory downstroke and measuring the angle between the third linear function and the horizontal.

15. A method according to claim 11, wherein extracting features of a breath waveform using the one or more transition points comprises fitting a quadratic function to the expiratory plateau, and determining a coefficient of the quadratic function.

16. A method according to claim 11, wherein extracting features of a breath waveform using the one or more transition points comprises fitting a hyperbolic tangent function to the expiratory upstroke and/or the inspiratory downstroke, and determining a coefficient of the hyperbolic tangent functions.

17. A method according to claim 1, the method further comprising obtaining a capnogram from a user, wherein the capnogram comprises a breath waveform.

18. An apparatus configured to perform a method according to claim 1.

19. A computer readable medium comprising instructions which, when executed by a processor, cause the processor to perform a method according to claim 1.

20. A method for training a machine learning model to learn an indicator of smoking history, the method comprising:
obtaining a plurality of breath waveforms from a plurality of capnograms;
extracting one or more features from the plurality of breath waveforms;
obtaining a label for each breath waveform indicating a corresponding smoking history; and
using the extracted features of the plurality of breath waveforms and the corresponding labels, training the machine learning model to learn the indicator of smoking history.
